SEQUENCE LISTING (2 sequences)

SEQ ID NO: 1 - protein, 416 aa, mus musculus
MLLSVPLLLGLLGLAAADPAIYFKEQFLDGDAWTNRWVESKHKSDFGKFVLSSGKFYGDLEKDKGLQTSQDARFYALSAKFEPFSNKGQTLVVQFTVKHEQNIDCGGGYVKLFPSGLDQKDMHGDSEYNIMFGPDICGPGTKKVHVIFNYKGKNVLINKDIRCKDDEFTHLYTLIVRPDNTYEVKIDNSQVESGSLEDDWDFLPPKKIKDPDAAKPEDWDERAKIDDPTDSKPEDWDKPEHIPDPDAKKPEDWDEEMDGEWEPPVIQNPEYKGEWKPRQIDNPDYKGTWIHPEIDNPEYSPDANIYAYDSFAVLGLDLWQVKSGTIFDNFLITNDEAYAEEFGNETWGVTKAAEKQMKDKQDEEQRLKEEEEDKKRKEEEEAEDKEDDDDRDEDEDEEDEKEEDEEESPGQAKDEL

SEQ ID NO: 2 - protein, 505 aa, mus musculus
MRFSCLALLPGVALLLASARLAAASDVLELTDENFESRVSDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASVVGFFRDLFSDGHSEFLKAASNLRDNYRFAHTNIESLVKEYDDNGEGITIFRPLHLANKFEDKTVAYTEKKMTSGKIKKFIQDSIFGLCPHMTEDNKDLIQGKDLLTAYYDVDYEKNAKGSNYWRNRVMMVAKKFLDAGHKLNFAVASRKTFSHELSDFGLESTTGEVPVVAIRTAKGEKFVMQEEFSRDGKALEQFLQEYFDGNLKRYLKSEPIPESNEGPVKVVVAENFDDIVNEEDKDVLIEFYAPWCGHCKNLEPKYKELGEKLSKDPNIVIAKMDATANDVPSPYEVKGFPTIYFSPANKKLTPKKYEGGRELNDFISYLQREATNPPIIQEEKPKKKKKAQEDL

(19) United States
(12) Patent Application Publication
(10) Pub. No.: US 2009/0004178 A1
(43) Pub. Date: Jan. 1, 2009

(54) PHARMACEUTICAL COMPOUND FOR BLOCKING THE CRT OR ERP57 TRANSLOCATION

(76) Inventor: Michel Sarkis OBEID, Paris (FR)

Correspondence Address: SAMUEL A. KASSATLY, 20690 View Oaks Way, SAN JOSE, CA, 95120, US

(21) Appl. No.: 11/845,063

(22) Filed: Aug. 25, 2007

Related U.S. Application Data
(63) Continuation-in-part of application No. 11/774,585, filed on Jul. 7, 2007.

(30) Foreign Application Priority Data
Sep. 8, 2006 (EP) 06291427.0-2107

(51) Int. Cl. A61K 39/395; A61P 37/00; A61K 38/02

(52) U.S. Cl. 424/130.1; 514/2

(57) ABSTRACT

A pharmaceutical compound is used in the treatment of a health condition in a mammal. The compound comprises an antibody that interferes with an increased translocation of a protein, leading to an increased immunogenicity of cell death. The antibody may be, for example, anti-calreticulin and/or anti-ERP57. This antibody includes any one or more of blocking or neutralizing antibody.

The present application further claims the priority of co-pending U.S. potent application Ser. No. 11/774,585, filed on Jul. 7, 2007, titled “Method, Apparatus, Compound, And Service For Effecting Localized, Non-Systemic, Immunogenic Treatment Of Cancer,” which is incorporated herein by reference in its entirety.

### PRIORITY CLAIM

The present application claims the priority of co-pending European patent application, Serial No. 06291427.0-2107, filed on Sep. 8, 2006, titled “Calreticulin For Its Use As A Medication For The Treatment Of A Disease Such As Cancer In A Mammal,” which is incorporated herein by reference in its entirety.

### FIELD OF THE INVENTION

The present invention generally relates to a method, an apparatus, a compound, a mammal, a test kit, a test chip, a medication, and a service for effecting localized, systemic and non-systemic, immunogenic treatment of a health condition or disease, such as cancer. More particularly, the present invention relates to the use of a protein, such as calreticulin and ERP57, to treat a health condition or disease in a mammal, such as cancer and fertility, etc.

### BACKGROUND OF THE INVENTION

Cancer is a major cause of mortality in numerous industrialized countries. Various methods of systemic cancer treatment such as surgery, immunotherapy, hormonotherapy, and chemotherapy, or local treatment such as radiotherapy, hove been used. Chemotherapy leads to the cell death. Two types of cell death are recognized: the apoptosis and the necrosis.

It has long been hypothesized that apoptotic cell death would be poorly immunogenic (or even tolerogenic), whereas necrotic cell death would be truly immunogenic. The difference between apoptotic cell death and necrotic cell death was believed to result from the intrinsic capacity of cells dying from non-apoptotic cell death to stimulate the immune response, for example by stimulating local inflammatory responses in response to danger signals and/or by triggering the maturation of dendritic cells (“DCs”).

In contrast to necrosis, which is characterized by a brisk plasma membrane rupture, apoptosis is associated with a series of subtle alterations in the plasma membrane that render the dying cells palatable to phagocytic cells. Apoptosis generates “eat me” signals that include the adsorption of soluble proteins from outside the cell (such as Clq and thrombospondin) and the translocation of molecules from inside the cell to the surface (such as phosphatidylserine, PS, and calreticulin (“CRT”)), as well, as the suppression of “don't eat me” signals (such as CD47) elicit the recognition and removal of apoptotic cells by professional and non-professional phagocytes. Suboptimal clearance of apoptotic cells can trigger unwarranted immune reactions and lead to autoimmune disease.

Nonetheless, it seems that the dichotomy between immunogenic necrosis versus tolerogenic apoptosis is an oversimplification. In addition, unscheduled (necrotic) tumor cell death might induce local immunosuppression. Moreover, the capacity of apoptotic tumor cells to trigger an immune response was determined to depend on the apoptosis inducer, leading to the identification of two morphologically undistinguishable subcategories of apoptosis, namely immunogenic and non-immunogenic apoptosis.

Several conventional chemotherapies induce non-immunogenic apoptosis. Thus, even after an initially efficient chemotherapy, patients might not develop an efficient antitumorous—immune response and are then overcome by chemotherapy-resistant tumorous variants.

The efficiency of a chemotherapy and the responsiveness of the tumors depend on the drugs and the molecules used in the chemotherapy. In general, the main drugs used in anti-tumorous chemotherapy could be divided into four groups: cytotoxic agents, hormones, immune response modulators, and inhibitors of the kinase tyrosin activity.

Cytotoxic agents include, for example, cytotoxic antibiotics such as anthracyclines (doxorubicin, idarubicin, mitoxantrone that are exemplary apoptosis inducers). However, prior to the advent of the present invention, anthracyclines were not believed to be capable of eliciting immunogenic cell death.

Numerous apoptosis inducers, including agents that target the endoplasmic reticulum (ER) (such as thapsigargin, tunicamycin, brefeldine A), mitochondria (such as arsenite, betulinic acid, C2 ceramide), proteasome (such as lactacystine, ALLN, MG132) or DNA (such as Hoechst 33343, camptothecin, etoposide, mitomycin C), failed to induce immunogenic apoptosis.

The mounting research in the field was not able, prior to the advent of the present invention, to identify the circumstances under which an immune response is triggered against dying tumor cells. Thus, prior to the advent of the present invention, the distinction between immunogenic and non-immunogenic cell death, as it relates to the biochemical change resulting from the cell surface membrane modification, remained unclear. In particular, there has been no association made between the presence of the calreticulin protein on the membrane surface of the cells and the immunogenic death of these cells, or the association between the absence of the calreticulin protein on the membrane surface of the cells and the non-immunogenic death of these cells.

Calreticulin was described for its ability to modulate the hormonal response, which is another conventional method for cancer treatment. Proteins that modulate hormone receptor induced gene transcription are present in the nucleus of the cell and either inhibit or promote the binding of a hormone to its receptor.

One exemplary method describes the use of the calreticulin that is present either in the endoplasmic reticulum of a cell or in the nucleus, and is limited to gene transcription and nuclear CRT. However, neither this nor other conventional methods have associated the alteration in the plasma membrane of the dying cells, namely the surface exposure of calreticulin, with the immunogenic cancer cell death, in order for an exogenous calreticulin or external provision of signals to confer immunogenicity to an otherwise non-immunogenic cell death, thus providing a desirable immunogenic, anti-cancer chemotherapy.

### SUMMARY OF THE INVENTION

The present invention satisfies this need, and presents a method, an apparatus, a compound, a mammal, a test kit, a test chip, a medication, a diagnostic tool, and a service of using the same (collectively referred to herein as “the invention” or “the present invention”) for effecting localized, non-systemic and systemic, immunogenic treatment of a health condition or disease in a mammal, such as cancer.

More particularly, the present invention teaches the use of calreticulin translocation to treat a health condition or disease in a mammal, such as cancer. As used herein, translocation generally describes the passage of matter (such as a protein) to the cell surface or to another desired location.

According to other embodiment of the present invention, the translocation of the protein ERP57 dictates the immunogenicity of the cancer cell death, in that the translocation of CRT depends on the translocation of ERP57.

According to still another embodiment of the present invention, a peptide can induce the translocation of CRT and ERP57. With this peptide, it would be possible to treat an established cancer condition. This peptide plays the role of a PP1/GADD34 inhibitor or any inducer of CRT and ERP57 translocation.

According to yet another embodiment of the present invention, a recombinant CRT or any of CRT translocation surface inducers or any mimetic form of calreticulin includes a truncated form of calreticulin or part or parts of calreticulin or calreticulin hybrids, exhibiting the same properties as the native form of calreticulin, may be used to treat a sterility condition in a mammal.

According to yet another embodiment of the present invention, a recombinant rEPR57 or any of rEPR57 translocation surface inducers or any mimetic form of rEPR57, includes a truncated form of rEPR57 or part or parts of rEPR57 or rEPR57 hybrids, exhibiting the same properties as the native form of rEPR57 may be used to treat a sterility condition in a mammal.

In one embodiment of the invention, the anthracyclines as cell death agent can also be used in the preparation of a medication for the treatment of a disease in a mammal, said medication inducing an increased location of calreticulin and/or ERP57 at the cellular surface.

The anthracyclines can also be used in the preparation of a medication for the treatment of a disease such as cancer or viral infection, etc., in a mammal, said medication promoting an induction of immunogenic apoptosis by increased calreticulin and/or ERP57 translocation at the cellular surface.

The present invention also deals with the use of anthracyclines in the preparation of a medication for the treatment of a disease such as cancer, viral infection or etc., in a mammal, said medication improving the efficiency of chemotherapy in a mammal in need of such chemotherapy by inducing an increased location of calreticulin and/or ERP57 at the cellular surface and/or an immunogenic apoptosis.

Moreover, the present invention concerns also a pharmaceutical composition which comprises an amount of an anthracyclines promoting an increased translocation of the calreticulin and/or ERP57 protein from the cytoplasm to the cell membrane which thus induces an immune response during apoptosis in a mammal.

The anthracyclines-comprised pharmaceutical composition promoting an increased translocation of the calreticulin and/or ERP57 from the cytoplasm to the cell surface can also improve chemotherapy response in a mammal.

The present invention also provides a method promoting the chemotherapy treatment response in a mammal including administration of the pharmaceutical composition comprising an amount of anthracyclines to a mammal in heed by inducing an increased location of calreticulin and/or ERP57 at the cellular surface and/or an immunogenic apoptosis. The anthracyclines could be, for example, doxorubicin, idarubicin or mitoxantrone, etc.

The present invention also concerns a product containing a chemotherapeutic agent and recombinant colreticulin and/or ERP57 as a combination product for its use in the treatment of disease.

The present invention further concerns a product containing a chemotherapeutic agent and the inhibitors (such as the catalytic subunit of the protein phosphatase 1 (PP1) inhibitor, the GADD34 inhibitor, the complex PP1/GADD34 inhibitor or the peptide inhibitor of the complex PP1/GADD34) as a combination product for its use in the treatment of disease. This combination product could be used for the treatment of a disease such as a cancer (such as breast cancer, prostate cancer, melanoma, colon cancer, etc.) or an infection (such as viral, bacterial, fungal or parasitic infection, etc.) or other conditions or diseases.

The present invention is also directed to a method for inducing increased calreticulin and/or ERP57 translocation from the cytoplasm to the cell surface, in order to enhance an immune response in the apoptosis phenomenon in a mammal. This method comprises administering pharmaceutically effective amount of an inhibitor as the catalytic subunit of the protein phosphatase 1 (PP1) inhibitor, the GADD34 inhibitor, the complex PP1/GADD34 inhibitor, or the peptide inhibitor of the complex PP1/GADD34. Preferably, the increased calreticulin and/or ERP57 translocation is from the cytoplasm to the membrane of tumorous cells. This method is intended to improve cancer treatment, preferably those tumors sensitive to VP16/etoposide, radiotherapy, or immunotherapy, e.g., melanoma, kidney cancer, colon cancer, breast or lung tumors, osteosorcoma, etc. Preferably, this method is directed to treat chemosensitive cancers as much as immunosensitive cancers.

The location of the calreticulin protein at the cell surface may be realized by antibodies anti-calreticulin which detects the endogenous form of calreticulin, the recombinant form, and the mimetic form. The present invention aims at the detection of various forms of the calreticulin protein at the cellular surface. This could be achieved in vitro, ex vivo, or in vivo.

Similarly, the location of the ERP57 protein at the cell surface may be realized by antibodies anti-ERP57 which detect the endogenous form of ERP57, the recombinant form, and the mimetic form. The present invention aims at the detection of various forms of the ERP57 protein at the cellular surface. This could be achieved in vitro, ex vivo, or in vivo.

The present invention also enables the induction of an increased translocation of calreticulin and/or ERP57 at the cellular membrane surface. The present invention uses the level of calreticulin and/or ERP57 translocation as a determining feature of anti-cancer immune responses, and as a decisive factor in the preparation of a treatment strategy for an immunogenic chemotherapy.

This method of detection of calreticulin and/or ERP57 at the cell surface could be used for predicting immunogenic apoptosis and also for therapeutic efficiency of a chemotherapy. The calreticulin and/or in these methods is used as a predictive marker of both immunogenic apoptosis and therapeutic efficiency of a chemotherapy. This method of quantitative detection can also be advantageous to predict risks of forced apoptosis that becomes too immunogenic. Inhibition of the translocation of the calreticulin and/or ERP57 at the cellular surface could decrease the immunogenicity of the calreticulin and thus reduce or alternatively block the immune response.

Thus, the present invention provides a method of detection of the calreticulin and/or ERP57 at the cell surface wherein the calreticulin and/or ERP57 at the cell surface is used as a predictive marker of immunogenic viral infection or autoimmune diseases or transplantation rejection/GVH disease or sign of fertility.

Additionally, in order to permit the detection of calreticulin and/or ERP57, the present invention also provides a kit for the detection of the calreticulin and/or ERP57 protein at the cell surface, according to the methods described herein. Such kit comprises at least anti-calreticulin and/or anti-ERP57 antibodies. In one embodiment of the invention, this detection kit could also be used for the quantitative detection of calreticulin and/or ERP57 at the cellular surface.

The present invention further concerns a method of detection of the calreticulin and/or ERP57 at the cellular surface for the screening of direct or indirect immunogenic drugs. Such screening method comprises detecting the calreticulin and/or ERP57 protein at the cell surface, and uses anti calreticulin antibodies and/or ERP57 antibodies for the screening of direct or indirect immunogenic drugs. The screening of direct and indirect immunogenic drugs could lead to the identification of more efficient anti-tumorous agents and new efficient molecules, for use in the treatment of mammal diseases and health-related conditions.

### BRIEF DESCRIPTION OF THE DRAWINGS

FIG. 1 is comprised of FIGS. 1A, 1B, and 1C, and illustrates the immunogenic cell death induced by anthracyclines, as follows:

FIG. 1A. Frequency of dead and dying cells after treatment with distinct chemotherapeutic agents. CT26 cells were cultured for 24 hours in the presence of the indicated agents for 24-48 h, and then were stained with Annexin V-FITC and the vital dye DAPI.

FIG. 1B. Identification of immunogenic cell death inducers. CT26 cells cultured as in FIG. 1A were injected into the left flank, followed by injection of life tumor cells in the right flank 8 days later. The percentage of tumor free mice was determined 120 days later as in FIG. 1C.

FIG. 1C. Incidence of tumors after inoculation of dying cells. The data show the actual frequency of tumor-free mice, for the experiment summarized in FIG. 1B. Day 1 was considered the day of inoculation of dying tumor cells, 1 week before challenge with dying tumor cells.

FIG. 1S is comprised of FIGS. 1SA, 1SB, 1SC, and 1SD, and illustrates the dissociation of CRT exposure and phosphatidyl serine exposure, as follows:

FIGS. 1SA, 1SB. Kinetics of CRT exposure. CT26 cells were treated with mitoxantrone for the indicated period, followed by immunofluorescence staining with a CRT-specific antibody and cytofluorometric analysis. Representative pictograms are shown in FIG. 1SA and quantitative data are reported in FIG. 1SB.

FIGS. 1SC, 1SD. Kinetics of PS exposure and cell death. Cells were cultured for the indicated period as illustrated in FIGS. 1SA and 1SB, followed by staining with Annexin V (which recognizes phosphatidylserin one the surface of dying cells) plus DAPI (which stains dead cells) and FACS analysis.

FIG. 2 is comprised of FIGS. 2A, 2B, 2C, 2D, and 2E, and illustrates the CRT surface exposure in immunogenic cell death, as follows:

FIGS. 2A through 2D. Identification of CRT as a surface-exposed molecule elicited by anthracyclines. Cells were treated for 4 h with doxorubicin alone (DX) or in combination with Z-VAD-fmk (DXZ), followed by biotinylation of the cell surface and purification of biotinylated proteins, 2D gel electrophoresis (FIG. 2A illustrates part of the gel at higher magnification) and mass-spectroscopic identification of one doxorubicin-induced spot as CRT (arrows in FIG. 2A and underlined peptides in the CRT protein sequence in FIG. 2B), immunoblot detection of CRT in the plasma membrane protein fraction or the total cell lysate (FIG. 2C) or immunofluorescence detection of CRT on the cell surface (in non-permeabilized live cells) or within the cell (after permeabilization and fixation) (FIG. 2D). The nuclei of untreated cells were visualized with Hoechst 33342 (blue), while those of doxorubicin-treated cells emit a red fluorescence (FIG. 2D). The circles in FIG. 2A indicate the position of ERP57.

FIG. 2E. Correlation between CRT exposure and immunogenicity. The surface exposure of CRT was determined by immunofluororescence cytometry while gating on viable (propidium iodine-negative) cells (inserts) and was correlated with the immunogenicity of cell death (as determined in FIG. 1). CO, control; Tg, thopsigorgin; Tu, tunicamycin.

FIGS. 2SA, 2SB, 2SC, 2SD illustrate the results of ERP57 surface exposure in immunogenic cell death, according to the present invention, as follows:

FIGS. 2SA, 2SB. Identification of ERP57 as a surface-exposed molecule elicited by anthracyclines. Cells were treated for 4 hours with doxorubicin alone (DX) or in combination with Z-VAD-fmk (DXZ), followed by biotinylation of the cell surface and purification of biotinylated proteins, 2D gel electrophoresis (FIG. 2SA illustrating port of the gel at high magnification), and mass-spectroscopic identification of one doxorubicin-induced spot as ERP57 (the arrows in FIG. 2SA and the underlined peptides in the CRT protein sequence in FIG. 2SB).

FIGS. 2SC, 2SD: Kinetics of ERP57 exposure. CT26 cells were treated with mitoxantrone for the indicated period, followed by immunofluorescence staining with a ERP57-specific antibody and cytofluorometric analysis. Representative pictograms are shown in FIG. 2SC and the quantitative data are reported in FIG. 2SD.

FIGS. 2SE and 2SF illustrate the Kinetics of phagocytosis and immunogenicity elicited by anthracyclines. CT26 cells were cultured for different periods with mitoxantrone or doxorubicin and then confronted with DC to measure their phagocytosis (FIG. 2SE), as in FIG. 3A or injected into mice, one week before challenge with live cells (FIG. 2SF). The numbers on each column of FIG. 2SF indicate the number of mice that were immunized.

FIG. 2SG illustrates the ERP57 exposure triggered by PP1/GADD34 inhibitors.

FIG. 3 is comprised of FIGS. 3A, 3B, 3C, 3D, 3E, and 3F and illustrates the requirement of surface CRT for phagocytosis of tumor cells by DC, as follows:

FIGS. 3A, 3B. Correlation between tumor cell phagocytosis and CRT exposure. Tumor cells labeled with Cell Tracker Orange were cultured with CD11c-expressing DC and the percentage of DC taking up tumor cells was determined (A) and correlated with the CRT surface exposure (B), measured as in FIG. 2E.

FIG. 3C. Blockade of CRT inhibits DC-mediated phagocytosis. Mitoxantrone-treated or control cells were incubated with a blocking chicken anti-CRT antibody, followed by detection of phagocytosis by CD.

FIGS. 3D, 3E, 3F. Knock-down of CRT inhibits DC-mediated phagocytosis and rCRT restores phagocytosis. Cells were transfected with the indicated siRNAs and optionally treated with rCRT, followed by immunoblot (FIG. 3D) detection of surface CRT (FIG. 3E) and phagocytosis by DC (FIG. 3F). Results are triplicates (X±SD) and representative of three independent experiments. * denotes statistically significant differences using the Student t′ test at p<0.001.

FIGS. 3SA, 3SB illustrate the results of ERP57 surface exposure in immunogenic cell death and that ERP57 is not implicated in the DC-mediated phagocytosis, according to the present invention, as follows:

FIG. 3SA. Correlation between tumor cell phagocytosis and exposure. Tumor cells labeled with Cell Tracker Orange were cultured with CD11c-expressing DC and the percentage of DC taking up tumor cells was determined (A) and correlated with the ERP57 surface exposure, measured as in FIG. 3A-3B

FIG. 3SB. Correlation between ERP57 exposure and immunogenicity. The surface exposure of ERP57 was determined by immunofluororescence cytometry while gating on viable (propidium iodine-negative) cells and was correlated with the immunogenicity of cell death (as determined in FIG. 2). CO, control; Tg, thapsigargin; Tu, tunicamycine (FIG. 3SB). The blockade of ERP57 did not inhibit DC-mediated phagocytosis. Mitoxantrone-treated or control cells were incubated with a blocking anti-ERP57 antibody, followed by the detection of phagocytosis by CD.

FIGS. 3SC and 3SD illustrate the importance of ERP57 for the translocation of calreticulin, and vice versa, as follows:

FIG. 3SC: Kinetics of CRT exposure. CT26 cells were treated with mitoxantrone for the indicated period, followed by immunofluorescence staining with a CRT-specific antibody and cytofluorometric analysis.

FIG. 3SC: Knock-down of ERP57 inhibits CRT translocation. Cells were transfected with the indicated ERP57 specific siRNA, treated with mitoxantrone for 4 h and followed by detection of surface CRT (FIG. 3SC). Similarly, Knock-down of CRT inhibits ERP57 translocation. Cells were transfected with the indicated CRT specific siRNA and followed by detection of surface ERP57.

FIG. 3SD: Knock-out of CRT inhibits ERP57 translocation. Wild type K41 cells lines and CRT-deficient K42 cells lines were treated with mitoxantrone for 4 h and followed by detection of surface ERP57 (FIG. 3SD). Similarly, Knock-down of ERP57 inhibits CRT translocation. Cells were transfected with the indicated ERP57 specific siRNA, treated with mitoxantrone for 4 h and followed by detection of surface calreticulin.

FIGS. 3SE and 3SF: Inhibitory profile of both CRT (FIG. 3SE) and ERP57 exposure (FIG. 3SF). Cells were treated with mitoxantrone or inhibitors of PP1/GADD34, after pre-incubation for 1 h with the indicated inhibitors of protein synthesis (cycloheximide), RNA synthesis (actinomycin D), microtubuli (nocodazol), or the actin cytoskeleton (latrunculin A). Then, CRT or ERP57 exposure was determined by immunocytofluorometry. Results are means of triplicates±SD for one representative experiment out of three.

FIG. 4 is comprised of FIGS. 4A, 4B, 4C, and 4D, and illustrates the CRT requirement for the immune response against dying tumor cells, as follows:

FIG. 4A. In vivo anti-cancer vaccination depends on CRT. CT26 colon cancer cells were transfected with the indicated siRNAs, then treated with rCRT and/or mitoxantrone (as in FIG. 3D) and the anti-tumor response was measured by simultaneously challenging BALB/c mice with mitoxantrone treated tumor cells in one flank and untreated, live tumor cells in the opposite flank.

FIG. 4B. Priming of T cell responses depending on CRT. CT26 tumor cells were left untransfected or transfected with the indicated siRNAs, then treated with medium alone, mitomycin C or mitoxantrone and injected into the right food pad of Balb/c mice. Five days later, mononuclear cells from the draining popliteal lymph nodes were challenged with freeze-thawed CT26 cells, and IFN-γ secretion was assessed at 72 hrs.

FIG. 4C. Exogenous supply of CRT enhances the immunogenicity of CRT-negative dying cells. CT26 cells lacking CRT expression after depletion of CRT with a siRNA and mitoxantrone treatment or after mitomycin treatment were coated with rCRT (inserts) and then injected into the food pad, followed by assessment of the IFN-γ secretion by cells from the draining lymph nodes as in FIG. 4B.

FIG. 4D. CRT-mediated amelioration of the immune response against etoposide or mitomycin C-treated tumor cells. CT26 cells were treated for 24 h with etoposide or mitomycin C (or PBS) and rCRT was optionally absorbed to the cell surface (inserts), followed by simultaneous injection of the etoposide or mitomycin C rCRT-treated tumor cells and live tumor cells in opposite flanks and monitoring of tumor growth.

FIG. 5 is comprised of FIGS. 5A-5G, and illustrates the induction of both calreticulin and ERP57 exposure and immunogenic cell death by inhibition of the PP1/GADD34 complex, as follows:

FIG. 5A. CRT exposure after anthracyclines treatment in the absence of a nucleus. Intact cells or enucleated cells (cytoplasts) were treated for 2 hours with mitoxantrone, followed by immunofluorescence detection of CRT exposure. Inserts show the effective removal of Hoechst 33342-stainable nuclei from the cytoplasts.

FIG. 5B. Phosphorylation of elF2a after treatment with anthracyclines. Cells were treated for four hours with mitoxantrone or doxorubicine followed by immunoblot detection of phosphorylated elF2a irrespective of its phosphorylation state and GAPDH as a loading control.

FIGS. 5C, 5D. Induction of both CRT and ERP57 exposure by knock-down of PP1. Cells were transfected with siRNAs specific for the indicated transcripts and were treated 36 h later for 2 h with mitoxontrone prior to immunoblot (FIG. 5C) and cell surface staining (FIG. 5D).

FIG. 5E. Kinetics of CRT and ERP57 exposure determine by FACS analysis after incubation of cells with the indicated agents.

FIG. 5F, 5G, PP1/GADD34 inhibitors render cell immunogenic via CRT. Tumor cells were first transfected with a control siRNA or a CRT-specific siRNA and then treated in vitro with etoposide, alone or in combination with PP1/GADD34 inhibitors. Two hours later, the surface CRT was detected to demonstrate the effective expression of CRT on control siRNA-transfected cells treated with etoposide alone or etoposide plus PP1/GADD34 inhibitors (FIG. 5F), and later, the cells were injected as in FIG. 1A to determine their capacity to inhibit the growth of live tumor cells inoculated one week later (FIG. 5G). The results represent the percentage of tumor free mice (comprising a total of 12 to 18 mice per group).

FIGS. 5SA, 5SB, and 5SC illustrate the inducement of the surface translocation of ERP57 and CRT by the peptide inhibitor of the complex PP1/GADD34, as follows:

FIG. 5SA: Kinetics of PS exposure and cell death. Cells were cultured as in FIG. 1SA and FIG. 1SB and treated with the inhibitory peptide of the complex PP1/GADD34 for the indicated period, followed by staining with Annexin V (which recognizes phosphatidylserin one the surface of dying cells) plus DAPI (which stains dead cells) and FACS analysis.

FIG. 5SB: Kinetics of ERP57 exposure. CT26 cells were treated with the inhibitory peptide of the complex PP1/GADD34 for the indicated period, followed by immunofluorescence staining with a ERP57-specific antibody and cytofluorometric analysis.

FIG. 5SC: Kinetics of CRT exposure. CT26 cells were treated with the inhibitory peptide of the complex PP1/GADD34 for the indicated period, followed by immunofluorescence staining with a CRT-specific antibody and cytofluorometric analysis.

FIG. 6 is comprised of FIGS. 6A, 6B, and 6C, and illustrates the therapeutic effect of CRT or PP1/GADD34 inhibitors injected into tumors. CT26 tumors established in immunocompetent wild type (FIG. 6A) or athymic nu/nu Balb/c mice (FIG. 6B) were injected locally with the indicated combinations of mitoxantrone, etoposide, mitomycin C, rCRT, salubrinal or tautomycin, followed by monitoring of tumor growth. Each curve represents one mouse. Numbers in the lower right corner of each graph indicate the number of mice that manifest complete tumor involution at day 45. FIG. 6C. Identical experimental setting using intratumoral etoposide plus contralateral subcutaneous injection of rec.CRT. The graphs depict one representative experiment out of two, comprising 5 mice/group.

FIG. 7 is comprised of FIGS. 7A, 7B, 7C, 7D, 7E, 7F, and 7G and illustrate the application of the invention to the process of mammal fertilization, as follows:

FIG. 7A illustrates the calreticulin surface exposure in capacitated sperms.

FIGS. 7B-7G illustrates the relationship between calreticulin exposure and sperm-egg fusion.

FIG. 8 illustrates a test kit or test chip for use in the implementation of the present invention.

FIG. 9 illustrates an overall method for the implementation of the methods of the present invention.

### DETAILED DESCRIPTION OF PREFERRED EMBODIMENTS

The present invention observes that the proteins (calreticulin and/or ERP57) exposure is present on cells that succumb to immunogenic cell death, yet locks on the surface of cells that undergo non-immunogenic cell death.

Two particular alterations were identified in the plasma membrane of dying cells: the surface exposure of calreticulin (CRT) and ERP57 which is the “chaperone” of CRT. This event only occurs in immunogenic cancer cell death. Exogenous CRT or the external provision of signals that induces CRT exposure confers immunogenicity to otherwise non-immunogenic cell death, allowing for an optimal anti-cancer chemotherapy.

Hence, the present invention concerns calreticulin and/or ERP57 for their use as a medication for the treatment of a disease in a mammal, which medication induces an increased location, including translocation of calreticulin and ERP57 at the cellular surface. Preferably, calreticulin and/or ERP57 may be used as a medication or treatment for cancer.

Cancers that might be treated by the methods of the present invention include, but not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma: leukemias, e.g., acute lymphocytic leukemia (myeloblastic, myelomonocytic, monocytic and erythroleukemia): and chronic lymphocytic leukemia; and polycythemia vera, lymphoma (Hodgkin's disease and non-Hodgkin's disease), multiple myeloma, Waldenstroms macroglobulinemia, and heavy chain disease.

The present invention also concerns the identification of calreticulin and/or ERP57 exposure as a determining feature of anti-cancer immune responses and delineates a strategy of immunogenic chemotherapy.

The location of the calreticulin and/or ERP57 at the cellular surface could be the result of the translocation of intracellular calreticulin and/or ERP57 to the cell surface or the result of the translocation of extracellular calreticulin and/or ERP57 to the cell surface. Thus, the present invention concerns calreticulin and/or ERP57 as a medication.

As used herein, calreticulin is available in an endogenous form, a recombinant form, or a mimetic form. The mimetic form of calreticulin includes a truncated form of calreticulin or part or parts of calreticulin or calreticulin hybrids, exhibiting the same properties as the native form of calreticulin, that is can be adsorbed or bound to the cellular membrane surface

As used herein, ERP57 is available in an endogenous form, a recombinant form, or a mimetic form. The mimetic form of ERP57 includes a truncated form of ERP57 or part or parts of ERP57 or ERP57 hybrids, exhibiting the same properties as the native form of ERP57, that is can be adsorbed or bound to the cellular membrane surface

The calreticulin and/or ERP57 translocation, either in an endogenous form, in a recombinant form, or in a mimetic form, results from the cytoplasm to the membrane of cells or from the extracellular medium to the membrane of the cells.

As used herein the mimetic form implies a truncated form of the calreticulin and/or ERP57 or parts of the calreticulin and/or ERP57 or hybrids thereof, exhibiting same properties as native form of calreticulin and/or ERP57 (i.e., location at the cellular surface).

Furthermore, according to the present invention, the calreticulin and/or ERP57 presence at a relatively high level at the cell surface renders the dying cells palatable to phagocytic cells such as dendritic cells. These cells interact with the immune system and then induce an immune response, that render the calreticulin and ERP57 as an inducer of immunogenic apoptosis.

Thus, the present invention concerns the use of calreticulin and/or ERP57 as a medication for the treatment of a disease (or an abnormal condition) in a mammal. Such medication would improve the efficiency of chemotherapy in a mammal in need of such chemotherapy by inducing an increased location of calreticulin and/or ERP57 at cell surface and/or induction of immunogenic apoptosis.

While the present invention is described in connection with specific proteins such as calreticulin and ERP57, and with specific reference to anthracyclines, inhibitors of the complex PP1/GADD34 and activators of the cited kinase activator it should be clear that the present invention is not limited to the use of these compositions and materials. It should be clear that any other immunogenic treatment e.g., oxaliplatin, paclitaxel (taxol), taxotere (Docetaxel), C16-ceramide, UVC light, gamma irradiation and the peptide PP1/GADD34 inhibitor, or other materials that are available or that may become available, may replace the anthracyclines.

The presence of calreticulin and/or ERP57 at the cellular surface could be the result of the translocation of intracellular calreticulin and/or ERP57 to the cell surface, or the result of the adsorption (addition) of extracellular calreticulin and/or ERP57 (e.g., recombinant CRT and/or ERP57) to the cell surface. The present invention discloses a treatment or medication, wherein the surface cell calreticulin and/or ERP57 could result either from the cytoplasm to the membrane of the cells or from an extracellular sources (e.g., recombinant) to the membrane of cells.

Furthermore, the present invention teaches that the calreticulin present in an increased amount (or level) at the cell surface renders the dying cells palatable to phagocytic cells, such as dendritic cells. These phagocytic cells of the host's (or patient's) immune system induce a systemic immune response. Thus, calreticulin behaves as an inducer of immunogenic apoptosis.

The present invention teaches the use of calreticulin and/or ERP57 as a medication that induces an immunogenic death (i.e. immunogenic apoptosis), for the treatment of a disease in a mammal. According to the present invention, calreticulin and/or ERP57 may be used as a medication for the treatment of various diseases, including for example, but not limited to cancer, such as breast cancer, prostate cancer, melanoma, colon cancer, etc., or an infection, such as viral, bacterial, fungal, or parasitic infection.

According to the present invention, calreticulin and/or ERP57 translocation may be used, not only as a treatment but also as an indicator for the success of a candidate treatment (such as chemotherapy) in a mammal. As a result, calreticulin and/or ERP57 translocation may be used as a tool for individualizing the treatment by selecting the most appropriate and effective treatment among numerous candidate treatments.

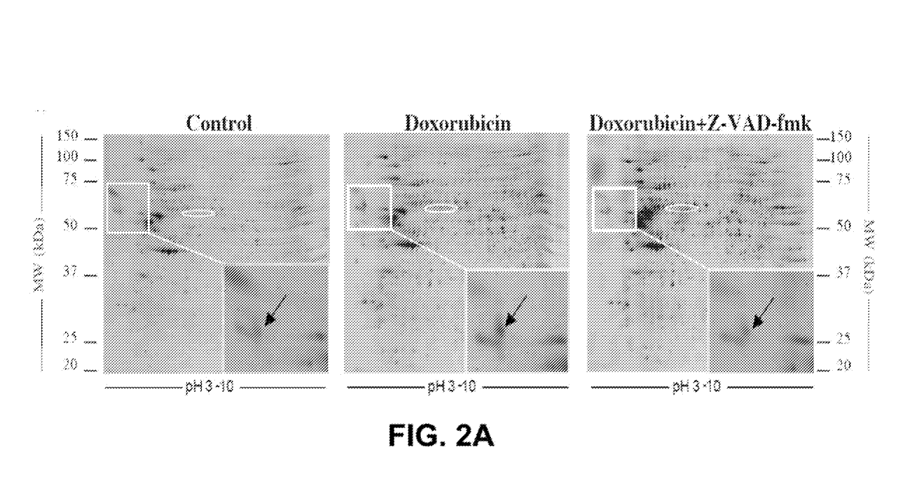

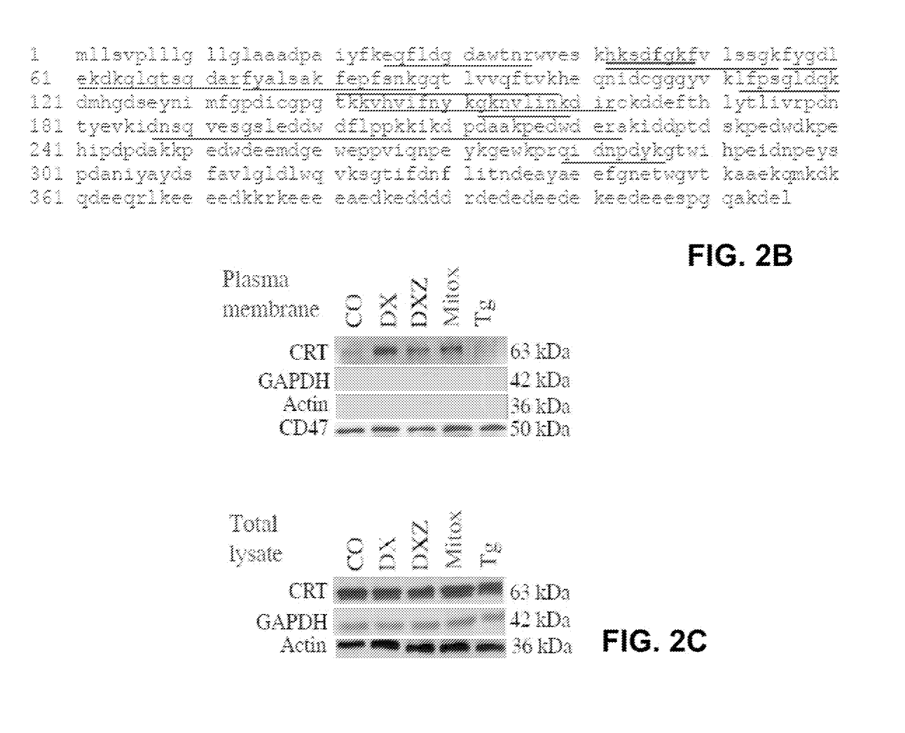

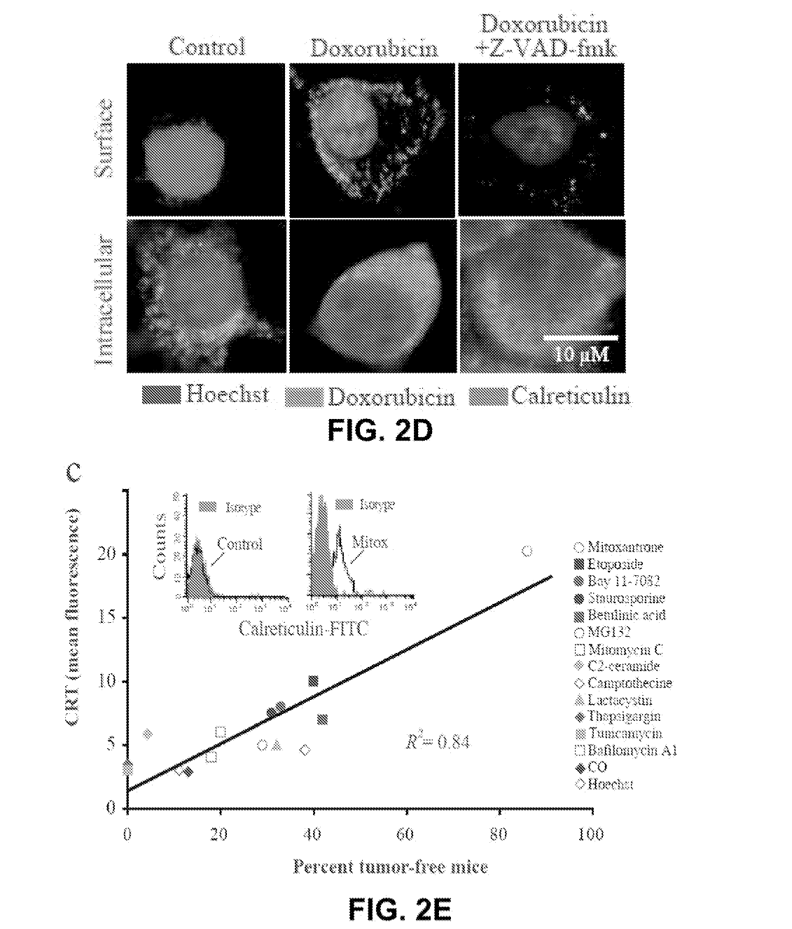

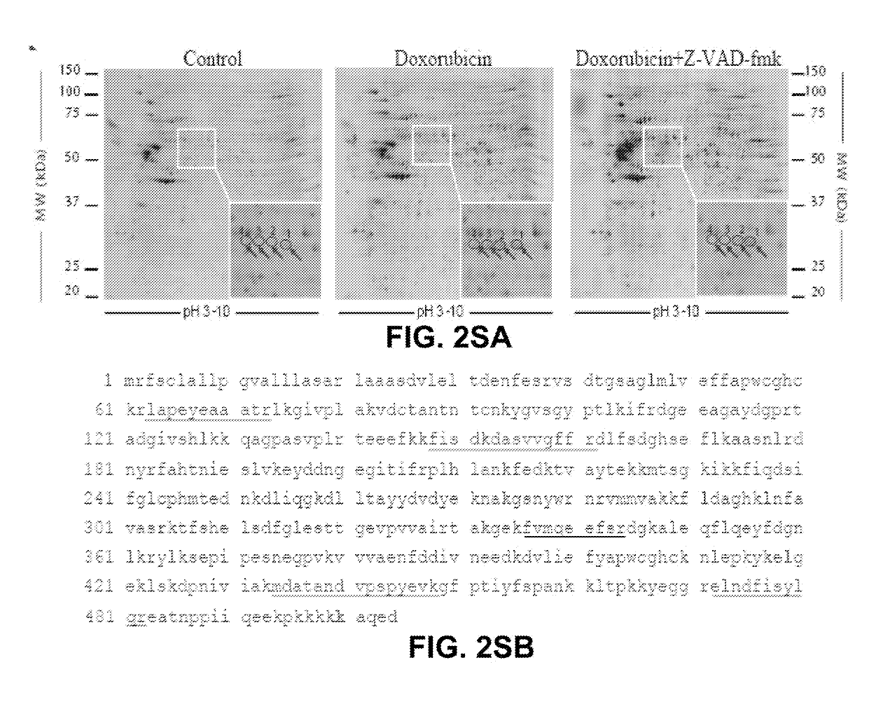

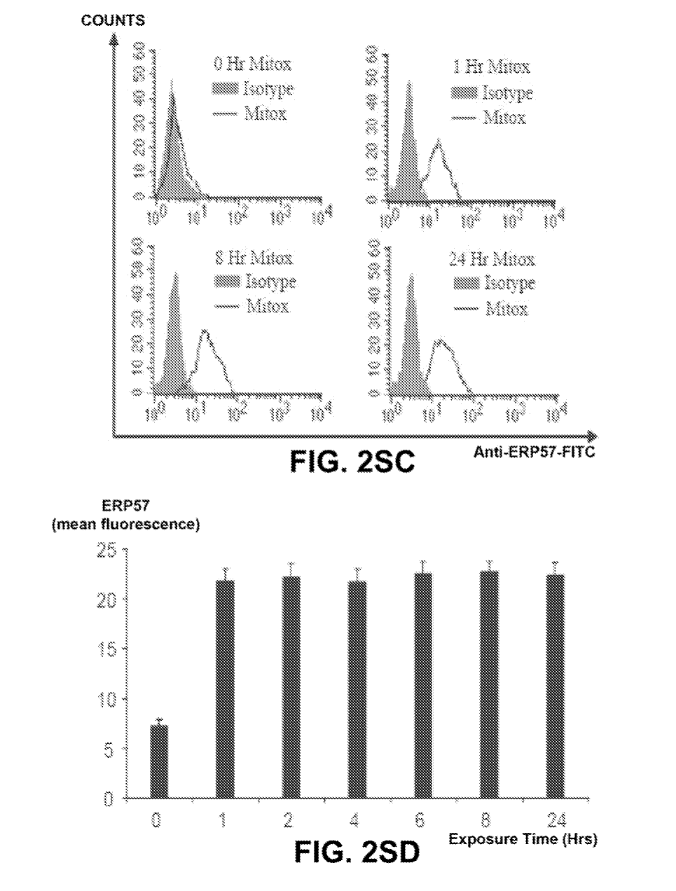

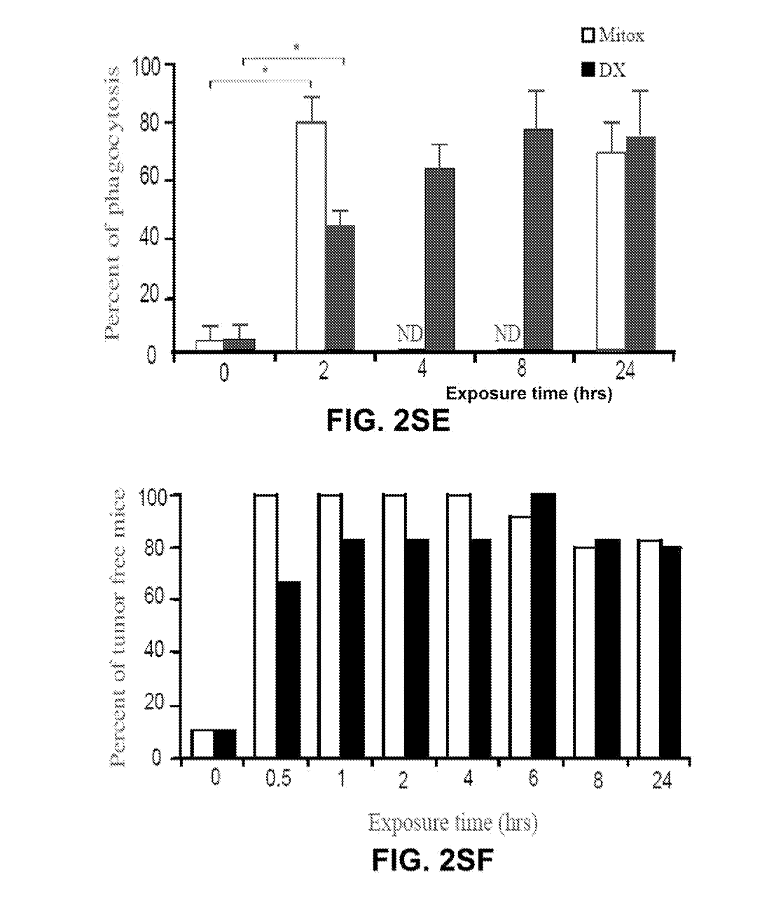

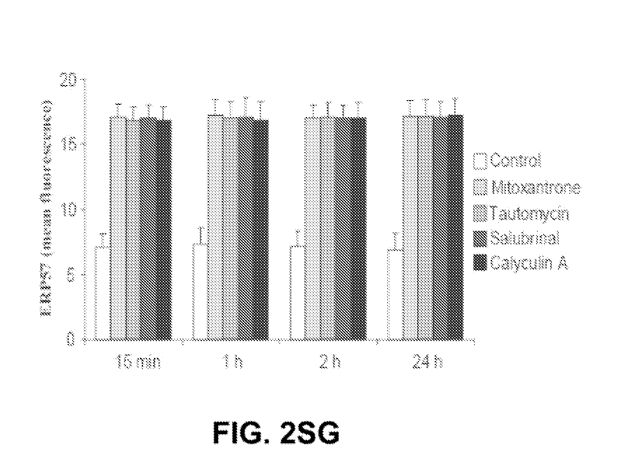

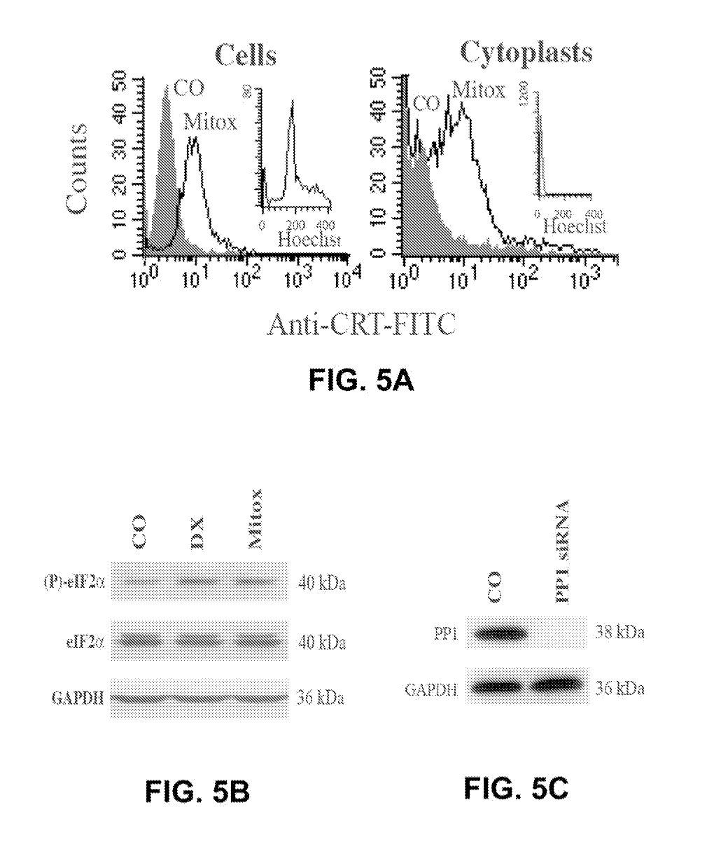

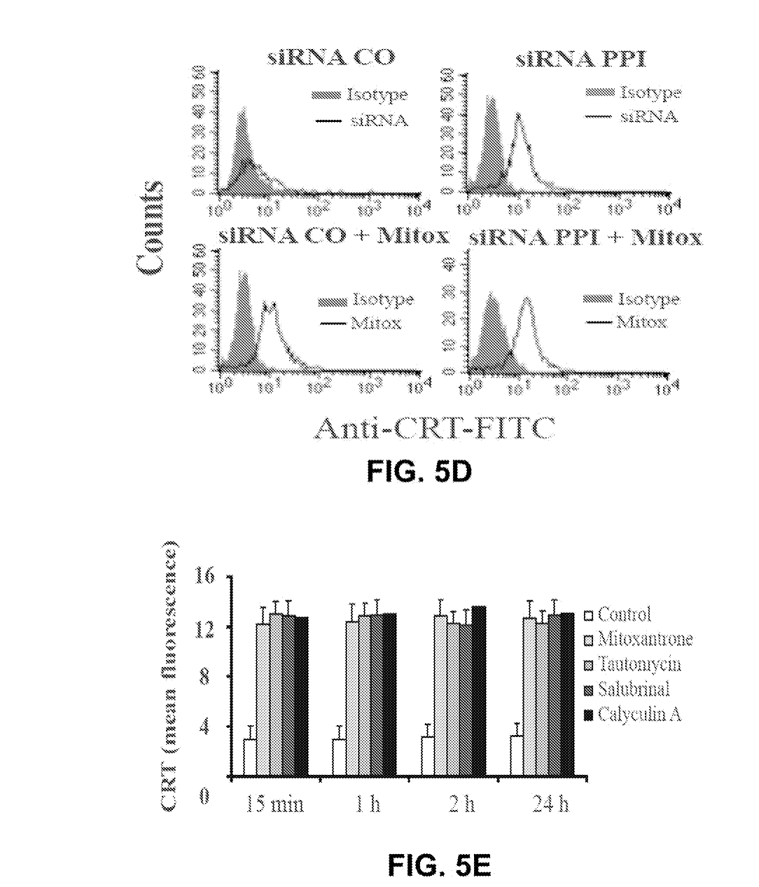

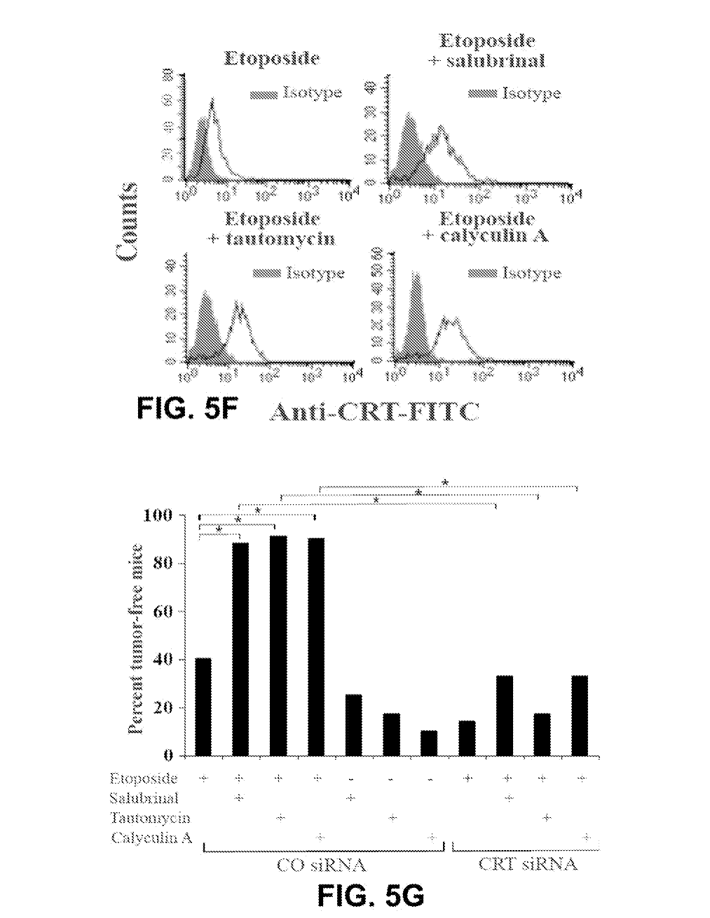

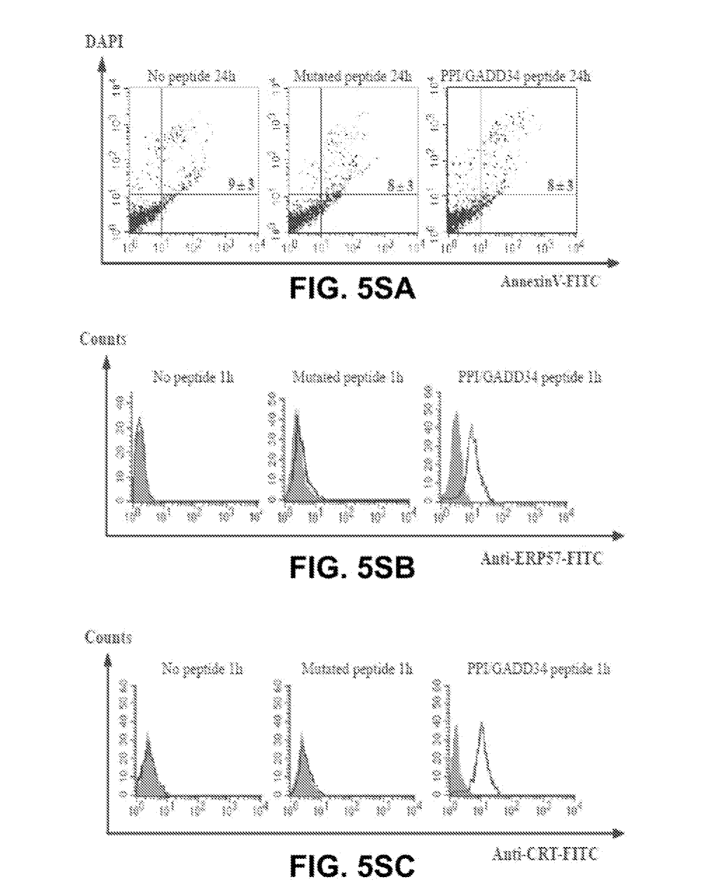

Calreticulin and/or ERP57 translocation exposure to the cell surface (or membrane) may be induced by various known or available methods, including but not limited to UVC light or irradiation, including for example, by not limited to gamma sources or any other immunogenic treatment e.g., oxaliplatin, paclitaxel (taxol), taxotere (Docetaxel), C16-ceramide, UVC light, gamma irradiation. In addition, calreticulin and/or ERP57, as illustrated in FIG. 2, including FIG. 2SG. Alternatively, translocation exposure might be triggered by anthracyclines, inhibitors such as PP1/GADD34 inhibitors, as illustrated in FIGS. 2, 5SA, 5SB, and 5SC.

According to the present invention, CRT translocation exposure might be triggered by a peptide inhibitor PP1/GADD34, as illustrated in FIG. 5SC. In addition, ERP57 exposure can be triggered by: anthracyclines (illustrated in FIGS. 2SC, 2SD), PP1/GADD34 inhibitors (illustrated in FIG. 2SG), and/or the peptide inhibitor PP1/GADD34 (illustrated in FIG. 5SB).

Such exposure involves the translocation of intracellular calreticulin and/or ERP57 to the cell surface through a molecular mechanism that involves the presence of both saturable calreticulin receptors on the cell surface that can bind exogenous (e.g., recombinant) calreticulin as well as endogenous purified calreticulin, and ERP57 saturable receptors on the cell surface that can bind exogenous ERP57 as well as endogenous purified ERP57.

More specifically, the present invention shows that the calreticulin protein is strongly (by, for example, a factor of 6) induced by anthracyclines (e.g., doxorubicin, mitoxantrone, idarubicine, etc.) (FIGS. 2A-2C) or other immunogenic treatment, for example: oxaliplatin, paclitaxel (taxol), taxotere (Docetaxel), C16-ceramide, gamma irradiation, UVC light and the peptide PP1/GADD34 inhibitor (FIGS. 5SA, 5SC). Immunoblot analyses of 2D gels and conventional electrophoreses of purified plasma membrane surface proteins (illustrated in FIG. 2C) confirmed the surface exposure of calreticulin after immunogenic treatment. This calreticulin surface exposure was also detectable by immunofluorescence staining of live cells (illustrated in FIG. 2D).

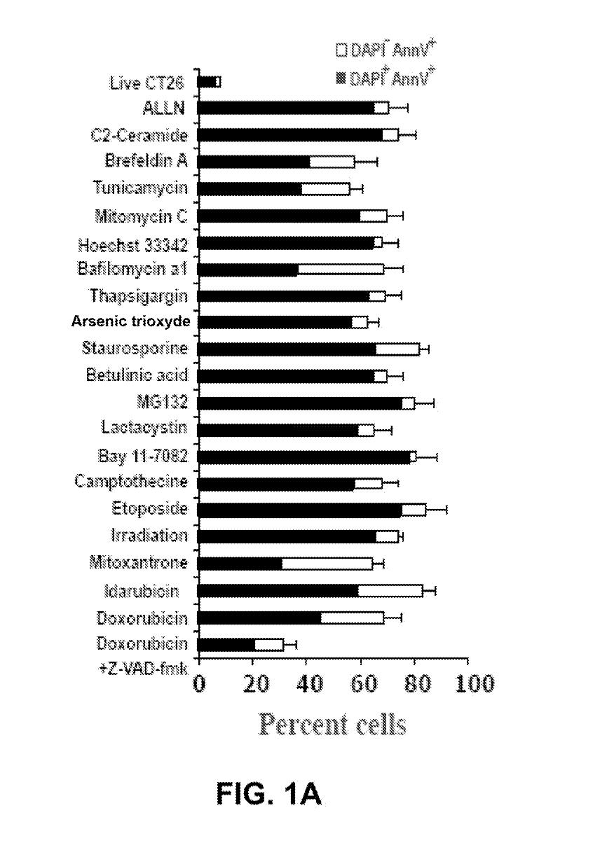

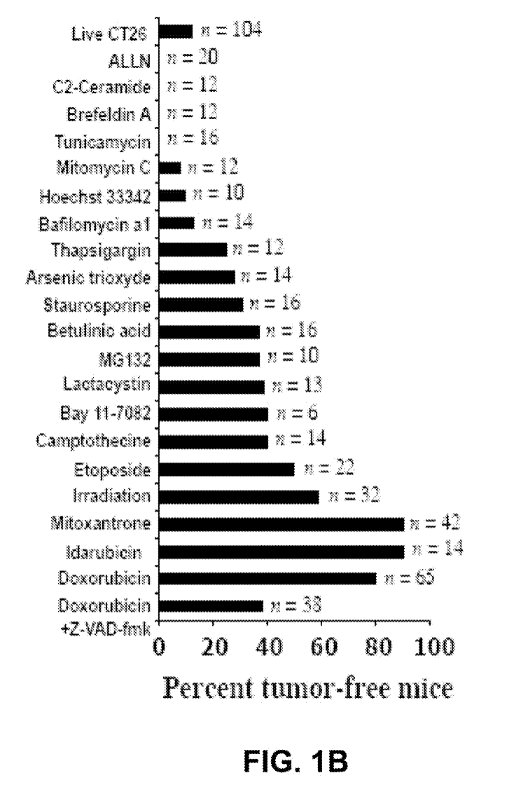

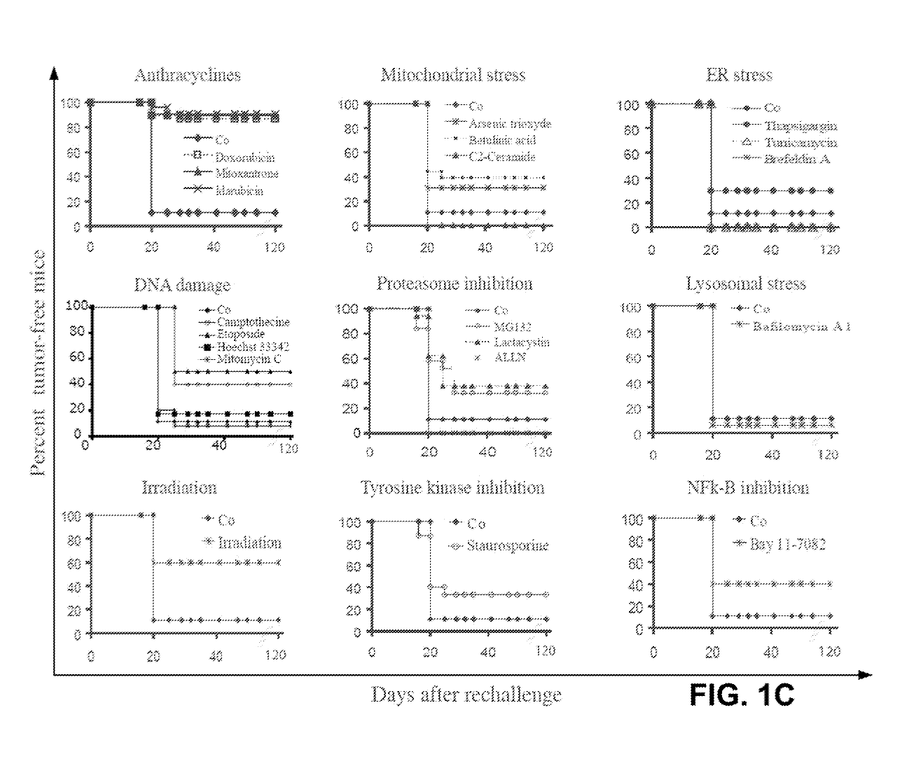

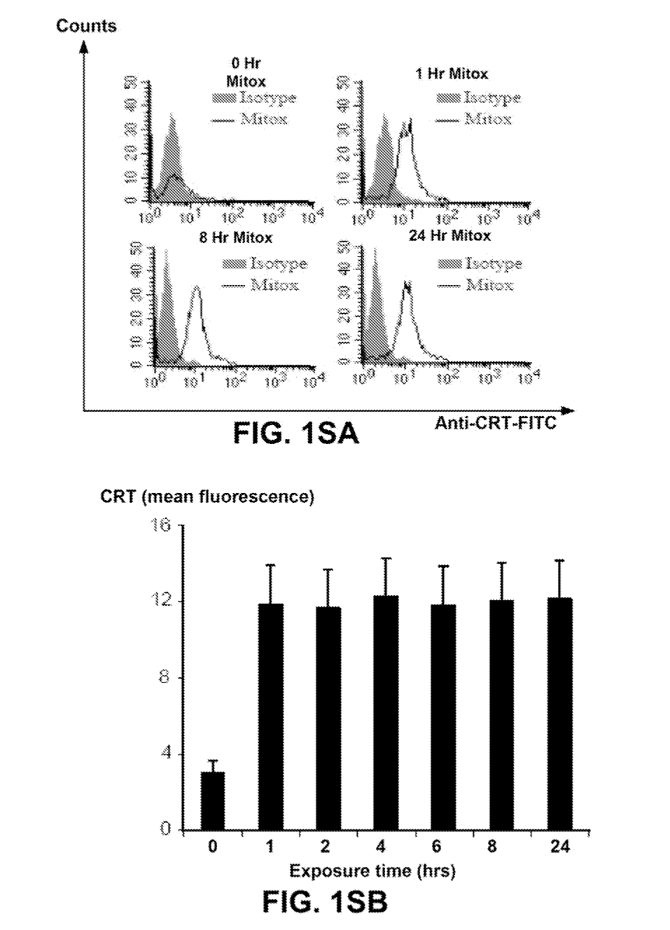

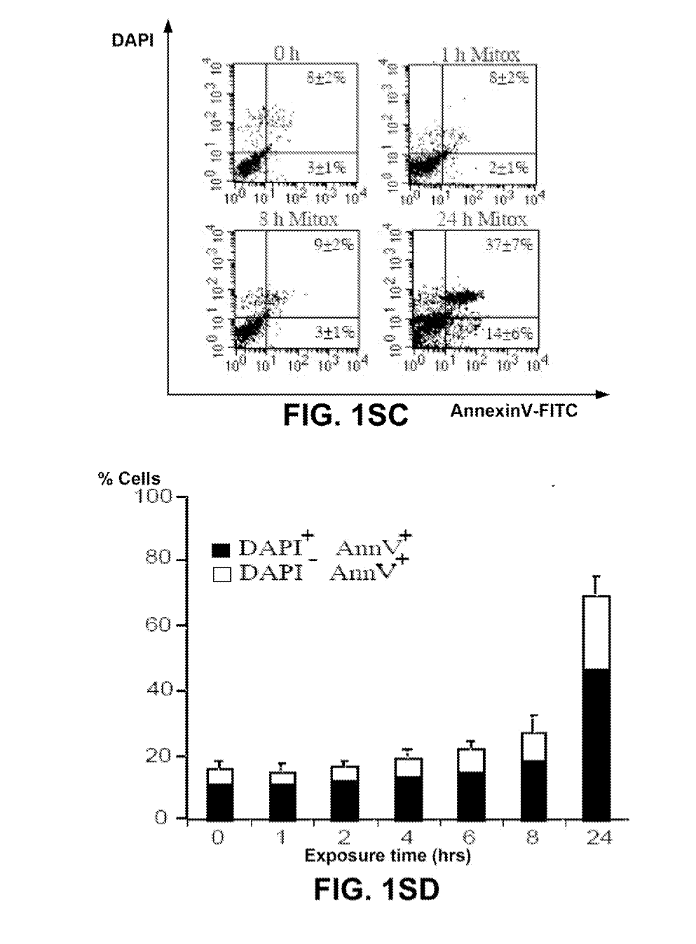

The induction of calreticulin exposure by immunogenic treatment, such as anthracyclines (e.g., doxorubicin, mitoxantrone, idarubicine, etc.), peptide inhibitor of PP1/GADD34 (FIGS. 1SA, 1SB, 5SC), UVC light and gamma irradiation, oxaliplatin, paclitaxel (taxol), taxotere (Docetaxel), C16-ceramide), could be a rapid process, detectable as soon as 1 hour following the treatment, and hence preceded the apoptosis associated phosphatidylserine (PS) exposure (FIGS. 1SC, 1SD, 5SA). Of note, there was a strong positive linear correlation (p<0.001) between the appearance of calreticulin at the cell surface (measured at 4 hours) and the immunogenicity elicited by the panel of 20 distinct apoptosis inducers (FIG. 2E).

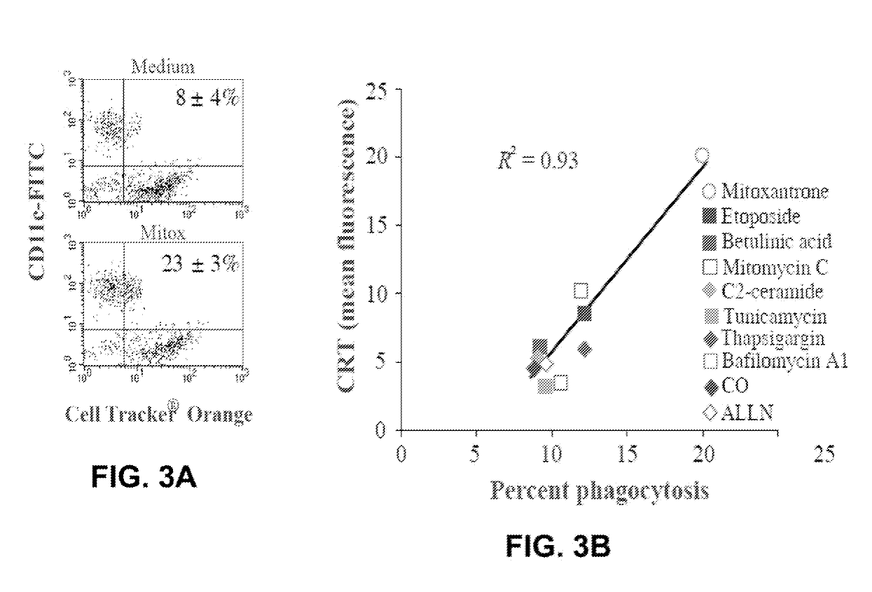

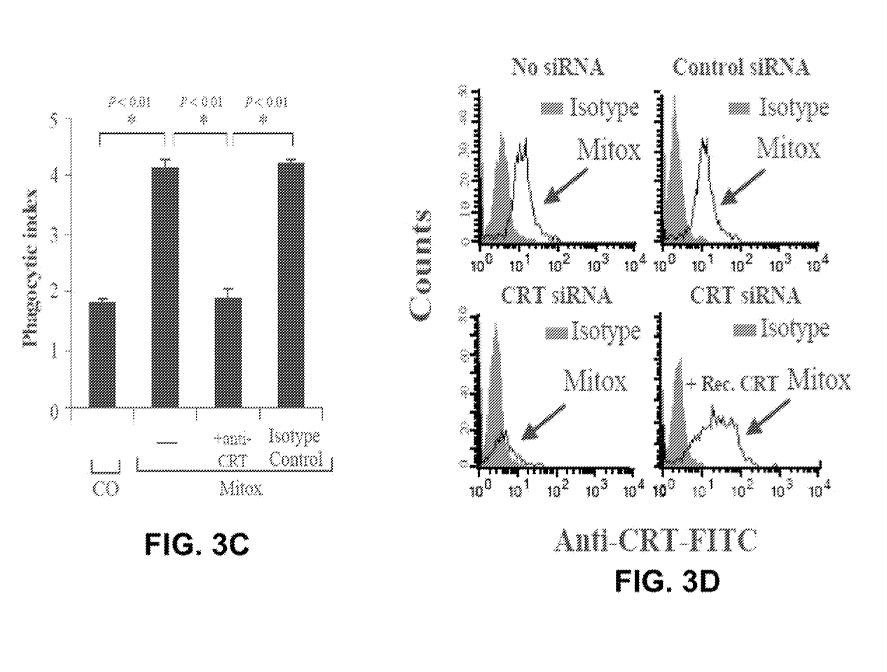

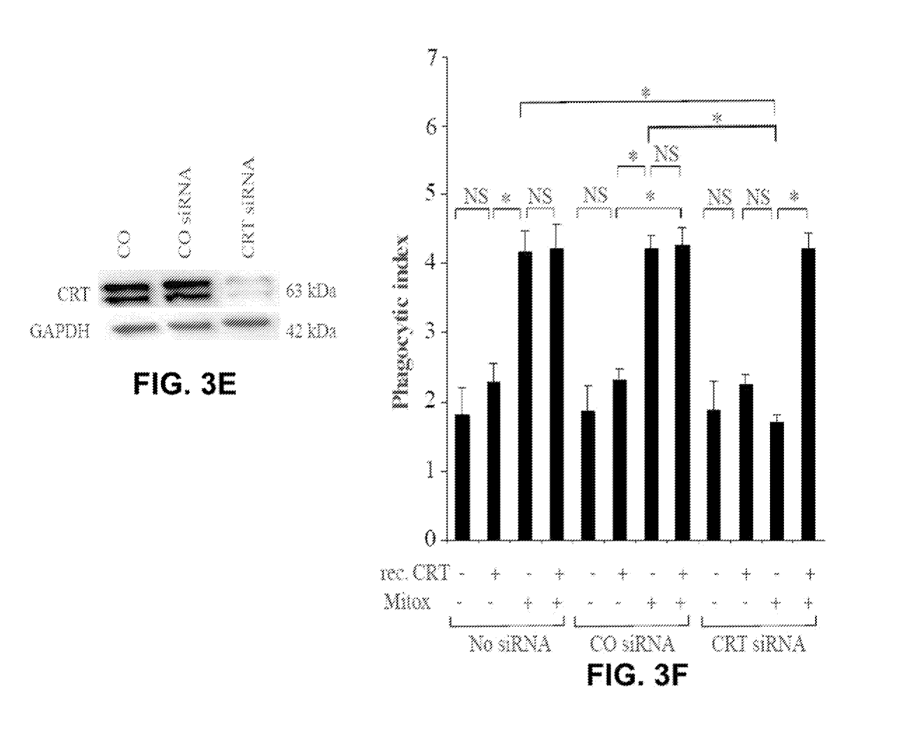

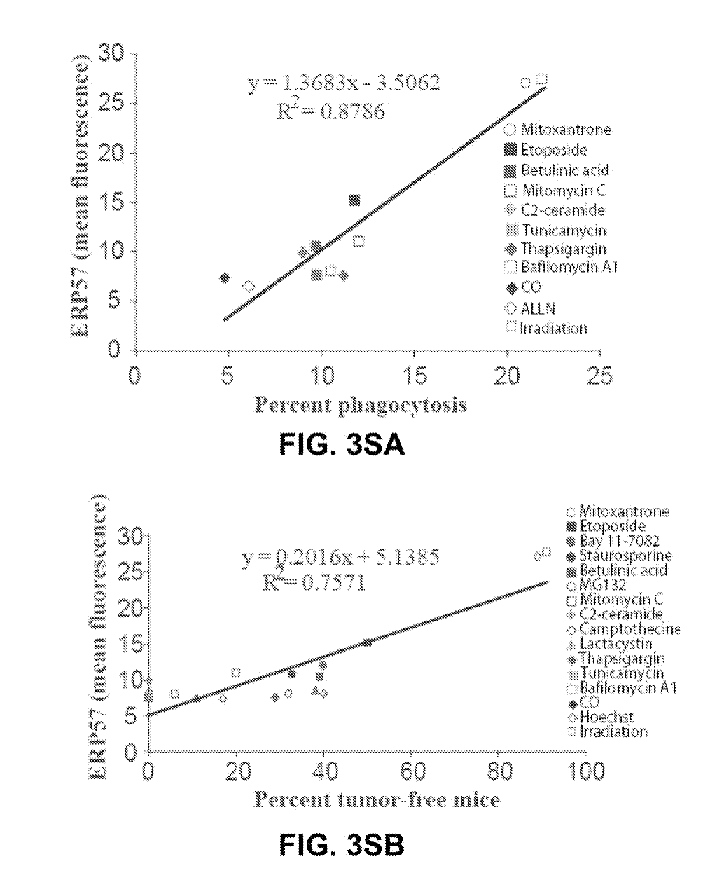

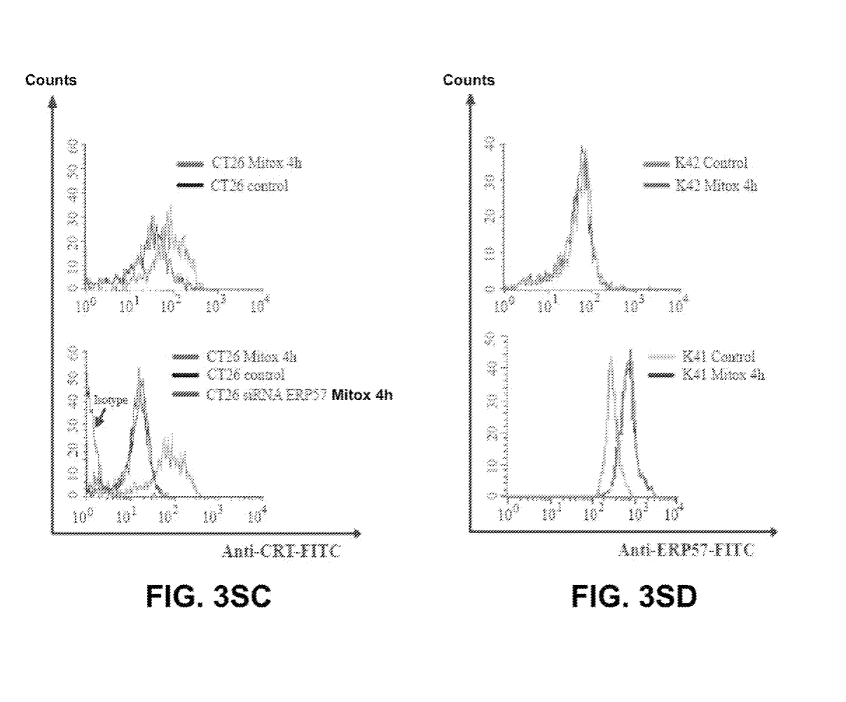

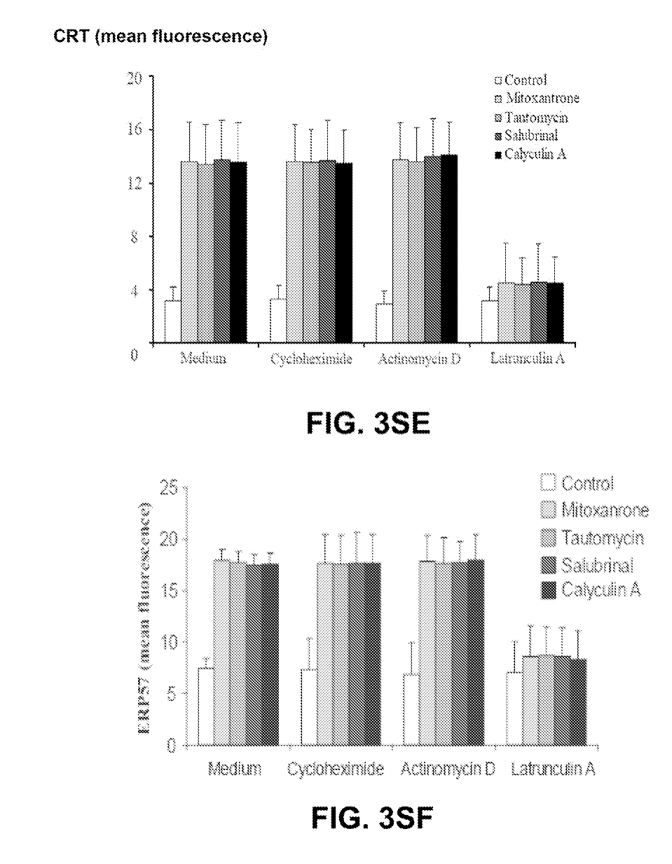

According to the present invention, the ERP57 protein could be strongly induced (by for example a factor between 4 and 8), by doxorubicin and anthracyclines in general (FIGS. 2SA and 2SB). Immunoblot analyses of 2D gels and conventional electrophoreses of purified plasma membrane surface proteins confirms the surface exposure of ERP57 after treatment with anthracyclines. This ERP57 surface exposure is detectable by immunofluorescence staining of anthracyclines-treated live cells. The induction of ERP57 exposure by anthracyclines, the peptide inhibitor of the complex PP1/GADD34, UVC light, gamma irradiation, oxaliplatin, paclitaxel (taxol), taxotere (Docetaxel) and C16-ceramide is a relatively rapid process, detectable as soon as 1 h after treatment (FIGS. 2SD, 5SB), and hence precedes the apoptosis associated phosphatidylserine (PS) exposure (FIGS. 1SC, 1SD, 5SA). There exists a strong positive linear correlation (p<0.001) between the appearance of ERP57 at the cell surface (measured at 4 h) and the immunogenicity elicited by the panel of 20 distinct apoptosis inducers (FIG. 3SB).

The translocation of CRT depends on the translocation of ERP57, and vice versa. The abolition of the protein ERP57 with specific siRNA blocks the translocation of CRT after mitoxantrone treatment (4 h) (FIG. 3SC). Moreover, the translocation of ERP57 was abolished in K42 cells line-deficient for CRT (FIG. 3SD). The suppression of the expression of CRT with specific siRNA blocks the translocation of ERP57. As a result, the translocation of CRT depends on the translocation of ERP57 and vice versa.

The immunogenicity and the immune response could be mediated by specific cells: the dendritic cells (“DC”). The present invention teaches that anthracyclines-treated tumor cells acquired a property to be phagocytosed by the dendritic cells a few hours following the treatment with doxorubicin or mitoxontrone, as illustrated in FIGS. 3A-3B and 2SE (similarly to the other immunogenic treatment), correlating with the rapid induction of calreticulin translocation, as illustrated in FIGS. 3B, 1SA, 1SB, and the acquisition of immunogenicity, such as for example, protection against the implantation of cancer tumor, as illustrated in FIGS. 2SF and 2E.

The immunogenicity and the immune response could be mediated by specific cells: the dendritic cells. According to the present invention, anthracyclines-treated tumor cells acquire the property to be phagocytosed by the dendritic cells, only a few hours following the treatment with doxorubicin or mitoxantrone (as illustrated in FIGS. 3A-3B, 2SE), correlating with the rapid induction of ERP57 (as illustrated in FIG. 3SA), and the acquisition of immunogenicity (as illustrated in FIG. 3SB).

### Materials and Methods

### Cell Lines and Cell Death Induction.

CT26 cells were cultured at 37° C. under 5% CO2 in RPMI 1640 medium supplemented with 10% FCS, penicillin, streptomycin, 1 mM pyruvate and 10 mM HEPES in the presence of doxorubicin (DX; 24 h, 25 mM), mitoxantrone (Mitox; 24 h, 1 mM, Sigma), idarubicin (24 h, 1 mM, Aventis, France), mitomycin C (30 mM, 48 h; Sanofi-Synthelabo, France), and/or zVAD-fmk (50 mM, 24 h: Bachem), tunicamycin (24 h, 65 mM), thapsigargin (24 h, 30 mM), brefeldin A (24 h, 50 mM, Sigma), etoposide (48 h, 25 μM, Tava classics), MG132 (48 h, 10 mM), ALLN (48 h, mM), betulinic acid (24 h, 10 mM), Hoechst 33343 (24 h, 0.2 mM), comptothecine (24 h, 15 mM), lactacystin (48 h, 60 mM), BAY 11-8072 (24 h, 30 mM), staurosporine (24 h, 1.5 mM), bafilomycin A1 (48 h, 300 nM), arsenic trioxide (24 h, 30 u mM), C2-ceramide (C2-C: 24 h, 60 mM), calyculin A (48 h, 30 nM), or tautomycin (48 h, nM, Sigma) and/or solubrinal (48 h, mM).

Inhibitory Peptide of the Complex PP1/GADD34

In some experiments, CT26 were cultured at 37° C. under 5% CO2 in RPMI 1640 medium supplemented with 10% FCS, penicillin, streptomycin, 1 mM pyruvate and 10 mM HEPES in the presence of 100 nm of the inhibitory peptide of the complex PP1/GADD34 or the mutated peptide. The sequence of the inhibitory peptide contains the protein transduction domain-5 (PTD-5), (RRQRRTSKLMKR), fused to the inhibitory sequence of the complex PP1/GADD34, (LKARKVRFSEKV). The mutated peptide sequence contains the protein transduction domain-5 (PTD-5), (RRQRRTSKLMKR), fused to the inhibitory mutated sequence of the complex PP1/GADD34, (LKARAVAFSEKV).

Cell Death Assays.

Cells were trypsinized and subjected to cytofluorometric analysis with a FACS Vantage after staining with 4,6-diamino-2-phenylindole (DAPI, 2.5 mM, 10 min, Molecular Probes) for determination of cell viability, and Annexin V conjugated with fluorescein isothiocyanate) for the assessment of phosphatidylserine exposure.

siRNAs and Manipulation of Surface CRT.

siRNA heteroduplexes specific for CRT (sense strand: 5′-rCrCrGrCrUrGrGrGrUrCrGrArArU rCrRrArATT-3′), GADD34 5′-(rCrArGrGrArGrCrArGrArUrCrArGrArUrArGrATT-3′), PP1 Ca (5′-rGrCrU rGrGrCrCrUrArUrArArGrArUrCrArGrATT-3′)), ERP57 (5′-rGrArGrGrCUUrGrCrCrCrCUrGrArGUrAU TT-3′ or an unrelated control (5′rGrCrCrGrGrUrArUrGrCrCrGrGrUrUrArArGrUTT-3′) were designed and synthesized by Sigma-Proligo. CT26 cells were transfected by siRNAs at a final concentration of 100 nM using HiPerFect. Thirty six hours post-transfection CT26 cells were assessed for total CRT content by immunoblotting. To restore CRT expression, cells were exposed to rCRT, produced as described, at 3 □g/10⁶ cells in PBC on ice for 30 min, followed by three washes.

Fluorescence Defection of Cell Surface CRT and ERP57.

CT26 cells (on a glass slide or in 12-well plates) were first washed with FACS buffer (1×PBS, 5% fetus bovine serum, and 0.1% sodium azide) and then incubated with rabbit anti-mouse CRT antibody (1:100, Stressgen), or rabbit anti-mouse ERP57 antibody (abcam) in FACS buffer at 4° C. for 30 min. Cells reacted with anti-rabbit IgG (H+L) Alexa fluor 488-conjugates (1:500) in FACS buffer at 4° C. for 30 min. After washing three times with FACS buffer, surface CRT and ERP57 was detected by cytofluorometric analysis on a FACS Vantage. In some experiments, cells were fixed with 4% paraformaldehyde, counterstained with Hoechst (2 μM; Sigma), followed by fluorescence microscopic assessment.

Immunoblot Analyses.

Cells were washed with cold PBS at 4° C. and lysed in a buffer containing 50 mM Tris HCl pH 6.8, 10% glycerol and 2% SDS. Primary antibodies detecting CRT (dilution 1/2000), CD47 (dilution 1/500), elF2α, elF2α-P, and PP1cα (dilution 1/2000), and GADD34 (dilution 1/2000), were revealed with the appropriate horseradish peroxidase-labeled secondary antibody and detected by ECL. Anti-actin or anti-GAPDH was used to control equal loading.

Anti-Tumor Vaccination and Treatment of Established Tumors.

All animals were maintained in specific pathogen-free conditions and all experiments followed the FELASA guidelines. 3×10⁶ treated CT26 cells were inoculated s.c. in 200 ml of PBS into BALB/c six-week-old female mice, into the lower flank, while 5×10⁵ untreated control cells were inoculated into the contralateral flank. For the tumorigenicity assay, 3×10⁶ treated or untreated CT26 cells were injected s.c. into nu/nu mice. To assess the specificity of the immune response against CT26, injections of either 5×10⁵ or 5×10⁶ of CT26 were made (for the mice immunized in a standard protocol or vaccination protocol, respectively). Tumors were evaluated weekly, using a caliper. In a series of experiments, BALB/c (wild type or nu/nu) carrying palpable CT26 tumors (implanted 14 days before for wild type or 7 days before for nu/nu mice by injection of 106 tumor cells) received a single intratumoral injection of 100 μM PBS containing the same concentration of anti-cancer agents and PP1/GADD34 inhibitors as those used in vitro, as well as rCRT (15 tag). For the assessment of local immune response, 3×10⁵ cells were injected in 50 μl into the footpad of mice. Five days later, mice were sacrificed and the draining lymph nodes were harvested. 1×10⁵ lymph node cells were cultured for 4 days alone or with 1×10⁴ CT26 cells killed by a freeze-thaw cycle in 200 μl in round-bottom 96-well plates. IFN-γ was determined by ELISA.

Generation of BMDCs.

BM cells were flushed from the tibias and femurs of BALB/c mice with culture medium composed of RPMA 1640 medium supplemented with 10% heat-inactivated FBS, sodium pyruvate, 50 mM 2-ME, 10 mM HEPES (pH 7.4), and penicillin/streptomycin. After one centrifugation, BM cells were resuspended in Tris-ammonium chloride for 2 min to lyse RBC. After one more centrifugation, BM cells (1×10⁶ cells/ml) were cultured in medium supplemented with 100 ng/ml recombinant mouse FLT3 ligand in 6-well plates. After 7 days, the non-adherent and loosely adherent cells were harvested with Versene, washed and transferred in 12-well plates (1.5×10⁶ cells/plate) for cocultures with tumor cells.

Phagocytosis Assays.

In 12-well plates, 25×10⁶ adherent CT26 cells were labeled with Celltracker Orange and then incubated with drugs. In some experiments viable CT26 were coated with 2 μg/10⁶ cells of chicken anti-CRT antibody (ABR affinity bioreagents) or an isotype control for 30 minutes prior to washing and feeding to dendritic cells Cs. Alternatively CT26 cells were coated with 3 μg/10⁶ cells of rCRT on ice for 30 minutes and washed twice prior to addition to dendritic cells. Cells were then harvested, washed three times with medium supplemented with FBS and cocultured with immature DC for 2 hours at a ratio of 1:1 and 1:5. At the end of the incubation, cells were harvested with Versene, pooled with non-adherent cells present in the supernatant, washed and stained with CD11c-FITC antibody. Phagocytosis was assessed by FACS analysis of double positive cells. Phagocytic indexes refer to the ratio between values obtained at 4° C. and values obtained at 37° C. of co-incubation between DC and tumor cells.

Statistical Analyses.

Data are presented as arithmetic means±standard deviation (SD) or percentages. The t-test was used to compare continuous variables (comparison of tumor growth), the Chi square test for non-parametrical variables (comparison of animal cohorts). For all tests, the statistical significance level was set at 0.05.

Biochemical Methods.

The purification of plasma membrane proteins, moss spectroscopy and the generation of cytoplasts are detailed below.

Biotinylation of GT26 Cell Surface Proteins.

Biotinylation and recovery of cell surface proteins were performed with a method adapted from Gottardi et al. (Gottardi, C. J. et al., “Biotinylation and assessment of membrane polarity: caveats and methodological concerns,” Am J Physiol 268, F285-295 (1995)) and Hanwell et al. (Hanwell, D. et al., “Trafficking and cell surface stability of the epithelial Na+ channel expressed in epithelial Madin-Darby canine kidney cells,” J Biol Chem 277, 9772-9779 (2002)). Briefly, 20×10⁶ CT26 cells grown on 75 cm² flask were placed on ice and washed three times with ice-cold PBS-Ca²⁺—Mg²⁺ (PBS with 0.1 mM CaCl2 and 1 mM MgCl2). Membrane proteins were then biotinylated by a 30-minute incubation at 4° C. with NHS-SS-biotin 1.25 mg/ml freshly diluted into biotinylation buffer (10 mM triethanolamine, 2 mM CaCl2, 150 mM NaCl, pH 7.5) with gentle agitation. CT26 cells were rinsed with PBS-Ca²⁺—Mg²⁺+glycine (100 mM) and washed in this buffer for 20 minutes at 4° C. to quench unreacted biotin. The cells were then rinsed twice with PBS-Ca²⁺—Mg²⁺, scraped in cold PBS, and pelleted at 2,000 rpm at 4° C. The pellets were solubilized for 45 min in 500 μl of lysis buffer (1% Triton X-100, 150 mM NaCl, 5 mM EDTA, 50 mM Tris, pH 7.5) containing protease inhibitors. The lysates were clarified by centrifugation at 14,000×g for 10 min at 4° C., and the supernatants were incubated overnight with packed streptavidin-agarose beads to recover biotinylated proteins. The beads were then pelleted by centrifugation, and aliquots of supernatants were taken to represent the unbound, intracellular pool of proteins. Biotinylated proteins were eluted from the beads by heating to 100° C. for 5 minutes in SDS-PAGE sample buffer before loading onto a 10% SDS-PAGE gel as described above. To ensure the absence of leakage of biotin into the cells, the absence of the intracellular protein actin and GAPDH in biotinylated extracts was systematically verified.

2D Gel Electrophoresis Analysis and Protein Identification by Mass Spectrometry.

Purified proteins were precipitated using the Ettan 2-D clean up kit were subsequently resuspended in urea buffer (7M urea, 2M thiourea, 2% Chaps, 1% Sulfobetaine SB3-10, 1% Amidosulfobetaine ASB14, 50 mM DTT). For the first dimension of protein separation, isoelectric focusing (IEF) was performed using 18-cm immobilized nonlinear pH gradient strips (pH 3 to 10; GE Healthcare) on a electrophoresis unit. Proteins (100 μg) were loaded by in-gel rehydratation for 9 h, using low voltage (30V) then run using a program in which the voltage was set for 1 h at 100 V, 2 h at 200 V, 1 h at 500 V, 1 h at 1,000 V, 2 hrs, 2 hrs voltage gradient 1,000-8,000V and 4 h at 8,000 V. Prior to the second-dimension electrophoresis, IPG gel strips were equilibrated for 10 min at room temperature in 1% dithiothreitol to reduce the proteins and sulfhydryl groups were subsequently derivatized using 4% iodoacetamide (both solutions were prepared in 50 mM Tris [pH 8.8]-6 M urea-30% glycerol-2% SDS-2% bromophenol blue). Strips were transferred to 1.0-mm-thick 10% (wt/vol) polyacrylamide gels (20 by 20 cm), and the second-dimension gels were run at 50 μA for 6 hours. Gels were stained with Sypro Ruby and visualized using a scanner. The analyser was used for matching and analysis of visualized protein spots among differential gels. Background subtraction was used to normalize the intensity value representing the amount of protein per spot.

Differentially expressed spots were excised from the gels with an automatic spot picker placed in Eppendorf tubes, and destained by washing for 5 min with 50 μL of 0.1 M NH4HCO3. Then 50 μL of 100 acetonitrile were added incubated for other 5 minutes. The liquid was discarded, the washing steps were repeated one more time and gel plugs were shrunk by addition of pure acetonitrile. The dried gel pieces were reswollen with 4.0 ng/μL trypsin in 50 mM NH4HCO3 and digested overnight at 37° C. Peptides were concentrated with ZipTip® μC18 pipette tips. Co-elution was performed directly onto a MALDI target with 1 μL of α-cyano-4-hydroxycinnamic acid matrix (5 μg/mL in 50% acetonitrile, 0.1% TFA). MALDI-MS and MALDI-MS/MS were performed on an analyzer with TOF/TOF ion optics. Spectra were acquired in positive MS reflector mode and calibrated either externally using five peaks of standard or internally using porcine trypsin autolysis peptide peaks (842.51, 1045.56 and 2211.10 [M+H]⁺ ions). Mass spectra were obtained from each sample spot by 30 sub-spectra accumulation (each including 50 laser shots) in a 750 to 4000 mass range. Five signal-to-noise best peaks of each spectrum were selected for MS/MS analysis. For MS/MS spectra, the collision energy was 1 keV and the collision gas was air.

MS and MS/MS data were interpreted using a software that acts as an interface between the database containing raw spectra and a local copy of a search engine. Peptide mass fingerprints obtained from MS analysis were used for protein identification in a non-redundant database. All peptide mass values are considered monoisotopic and mass tolerance was set <50 ppm. Trypsin was given as the digestion enzyme, 1 missed cleavage site was allowed, methionine was assumed to be partially oxidized and serine, threonine and tyrosine partially phosphorylated. Scores greater than 71 were considered to be significant (p<0.005). For MS/MS analysis, all peaks with a signal-to-noise ratio greater than 5 were searched against the database using the same modifications as the MS database. Fragment tolerance less than 0.3 Do was considered.

Preparation of Cytoplasts.

Trypsinized CT26 cells were enucleated as described. Briefly, cells were treated in 2 ml of complete RPMI medium containing cytochalosin B (10 μg/ml: Sigma) and DNase I (80 U/ml; Sigma). Cell suspension was adjusted to a final concentration of 5×10⁶/ml and incubated at 37° C. for 45 minutes before being layered onto a previously prepared discontinuous Ficoll density gradient (3 ml of 100%, in 1 ml of 90% and 3 ml of 55% Ficoll Paque layer containing 5 μg/ml cytochalasin B and 40 U/ml DNase I; gradients were prepared in ultracentrifuge tubes and pre-equilibrated at 37° C. in a CO2 incubator overnight). Gradients containing cell suspensions were centrifugated in a prewarmed SW41 Beckman rotor at 25 000 rpm for 20 minutes at 30° C. The cytoplasts-enriched fraction was collected from the interface between 90 and 100% Ficoll layers, washed in complete RPMI 1640 medium, and incubated at 37° C. The cells were incubated with mitoxantrone (MTX), calyculin (CA), salubrinal (Sol) and tautomycin (TA) for the period of time indicated in the experiment. Then the cell surface CRT was detected (see materials and methods) and the viability was determined by with propidium iodine staining (2 μg/ml, Sigma) for 5 min followed by cytofluorometric analysis. Alternatively cythoplasts were cocultured with immature DC for 2 hours at a ratio of 1:1 and 1:5. At the end of the incubation, cells were harvested with versene, pooled with non-adherent cells present in the supernatant, washed and stained with CD11c-FITC antibody. Phagocytosis was assessed by FACS analysis of double positive cells.

The following examples provide some illustrations of the present invention.

### Example 1

### CRT Exposure Defines Immunogenic Cell Death

Dying CT26 tumor cells exposed to a panel of −20 distinct apoptosis inducers (all of which induced −70±10% apoptosis, as determined by double staining with the vital dye DAPI and the PS-binding dye Annexin V, FIG. 1A) were injected into one flank of immunocompetent BALB/c mice, followed by rechallenge of the animals with live tumor cells injected into the opposite flank 8 days later. Protection against tumor growth then was interpreted as a sign of anti-tumor vaccination (FIG. 1B) because such protection was not observed in athymic (nu/nu) BALB/c mice. Most apoptosis inducers, including agents that target the endoplasmic reticulum (ER) (thapsigargin, tunicamycin, brefeldin), mitochondria (arsenite, betulinic acid, C2 ceramide), proteasome (ALLN, MG132, lactacystin) or DNA (Hoechst 33342, camptothecin, etoposide, mitomycin C), failed to induce immunogenic apoptosis, while anthracyclines (doxorubicin, idarubicin and mitoxantrone) elicited immunogenic cell death (FIG. 1B, C). To identify changes in the plasma membrane proteome, biotinylated surface proteins were affinity-purified from cells that were either untreated or short-term (4 h) treated with doxorubicin or doxorubicin plus Z-VAD-fmk, a pan-caspase inhibitor that reduces the immunogenicity of doxorubicin-elicited cell death (FIG. 1B). Comparison of 2D electrophoreses (FIGS. 2A and 2SA), followed by mass spectroscopic analyses, led to the identification of CRT (FIG. 2B) and ERP57, spots 1, 2, 3 and 4 (FIG. 2SB) as a protein that was strongly induced by doxorubicin (by a factor of 6 for CRT, a factor of 4.1 for spot 1 of ERP57, a factor of 3.4 for spot 2 of ERP57, a factor of 8 for spot 3 of ERP57, and a factor of 8.1 for spot 4 of ERP57), but less so by a factor of 1.8 for CRT, a factor of 2.2 for spot 1 of ERP57, a factor of 1.7 for spot 2 of ERP57, a factor of 1.2 for spot 3 of ERP57, and a factor of 1.5 for spot 4 of ERP57, by doxorubicin combined with Z-VAD-fmk. The different spots of ERP57 correspond to the different status of phosphorylation.

The protein ERP57 is a CRT-interacting chaperone. Immunoblot analyses of 2D gels and conventional electrophoreses of purified plasma membrane surface proteins confirmed the surface exposure of CRT (FIG. 2C) and ERP57 after treatment with anthracyclines. The CRT (FIG. 2D) and ERP57 surface exposure was also detectable by immunofluorescence staining of anthracyclines-treated live cells and was not accompanied by a general increase in the abundance of intracellular CRT or ERP57 (FIGS. 2C, 2D).

The ERP57 surface exposure was also detectable by immunofluorescence staining of anthracyclines-treated live cells and was not accompanied by a general increase in the abundance of intracellular ERP57. The induction of CRT and ERP57 exposure by anthracyclines was a rapid process, detectable as soon as 1 hour after treatment (FIGS. 1SA, 1SB, 2SC, 2SD), and hence preceded the apoptosis-associated phosphatidylserine (PS) exposure (FIGS. 1SC, 1SD). It should be noted that CRT exposure is correlated with ERP57 exposure. In contrast, CRT or ERP57 exposure did not correlate with alterations in CD47 expression (FIG. 2C). Of note, there was a strong positive linear correlation (p<0.001) between the appearance of CRT at the cell surface (measured at 4 hours) and the immunogenicity elicited by the panel of 20 distinct apoptosis inducers (FIG. 2E), and also between the appearance of ERP57 at the cell surface (measured at 4 hours) and the immunogenicity elicited by the panel of 20 distinct apoptosis inducers exposure (FIG. 3SB).

### Example 2

### The Importance of ERP57 is Critical for the Translocation of CRT, and the Importance of CRT is Critical for the Translocation of ERP57

The knockdown of ERP57 with specific siRNA suppressed the translocation of CRT (FIG. 3SC). Moreover, the translocation of ERP57 is suppressed in CRT-deficient k42 cell lines (FIG. 3SD). This translocation was not affected in wild type K41 cells lines (FIG. 3SD). Similarly, the translocation of ERP57 was abolished in CT26 transfected with specific siRNA for CRT. Hence, the presence of ERP57 is critical for the translocation of CRT and the presence of CRT is critical for the translocation of ERP57.

### Example 3

### Requirement of CRT and not ERP57 for DC-Mediated Recognition of Dying Tumor Cells

In view of the established role of CRT as an “eat me” signal it was decided to further investigate the possible implication of CRT in the phagocytosis of anthracyclines-treated tumor cells by DC, a cell type that is stringently required for mounting an immune response against apoptotic tumor cells. Anthracyclines-treated tumor cells acquired the property to be phagocytosed by DC quickly, well before the manifestation of apoptotic changes, within a few hours after treatment with doxorubicin or mitoxantrone (FIGS. 3A, 2SE), correlating with the rapid induction of CRT (FIGS. 3B, 1SA, 1SB) and the acquisition of immunogenicity (FIG. 2SF), and correlating with rapid induction of ERP57 (FIGS. 2SD, 3SB).

The presence of CRT and/or ERP57 on the surface of tumor cells treated with a panel of distinct cell death inducers strongly correlated with their DC-mediated phagocytosis, suggesting that CRT and ERP57 are important in mediating the uptake of tumor cells by DC (FIGS. 3B, 3SA). Accordingly, blockade of the CRT present on the surface of mitoxantrone-treated cancer cells by means of a specific antibody from avian origin (which cannot interact with mouse Fc receptors) inhibited their phagocytosis by DC (FIG. 3C). In contrast, blocking the surface ERP57 with a specific antibody did not affect the efficiency of the phagocytosis of CT26 tumor cells by DC.

Similarly, knockdown of CRT with a specific siRNA (FIGS. 3D, 3E) suppressed the phagocytosis of anthracyclines-treated tumor cells (FIG. 3F). Moreover, the knockdown of ERP57 with specific siRNA suppressed the translocation of CRT (FIG. 3SC) and the phagocytosis of CT26 by DC. Addition of recombinant CRT protein (rCRT), which binds to the surface of the cells, could reverse the defect induced by the CRT-specific siRNA or ERP57 specific siRNA, both at the level of CRT expression (FIG. 3D) and phagocytosis by DC (FIG. 3F). In contrast, the addition of rERP57 did not reverse the defect induced by the CRT-specific siRNA or ERP57 specific siRNA at the level of CRT expression and phagocytosis by DC. Of note, rCRT alone or ERP57 alone could not promote DC maturation ex vivo over a large range of concentrations. Hence, surface CRT and not ERP57 elicits phagocytosis by DC.

### Example 4

### Requirement of CRT and not ERP57 for Immunogenicity of Dying Tumor Cells

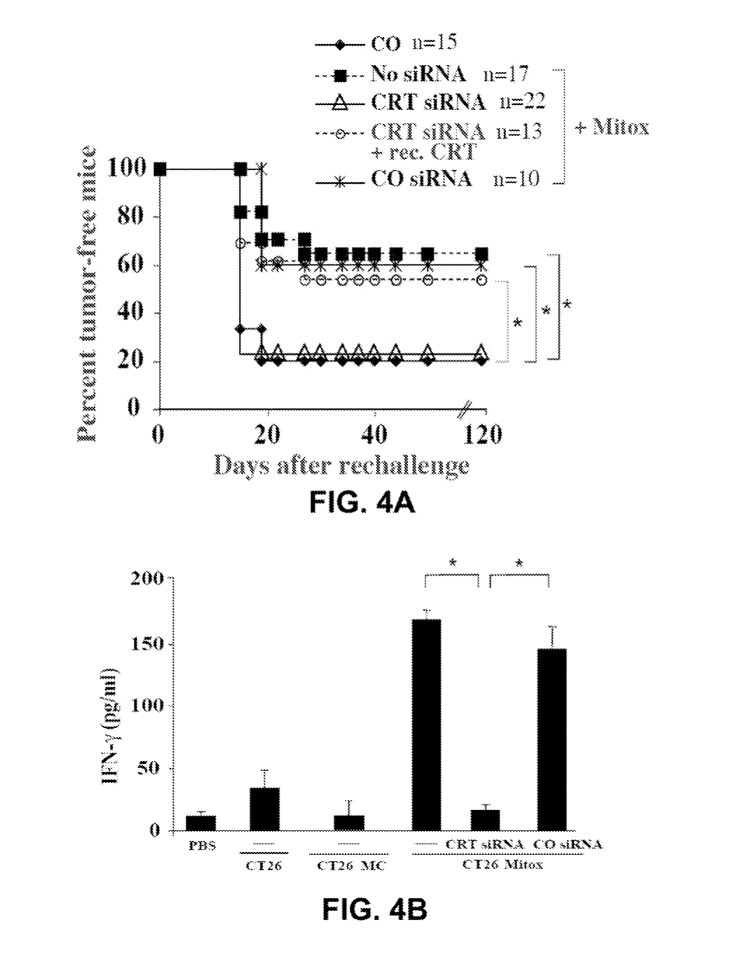

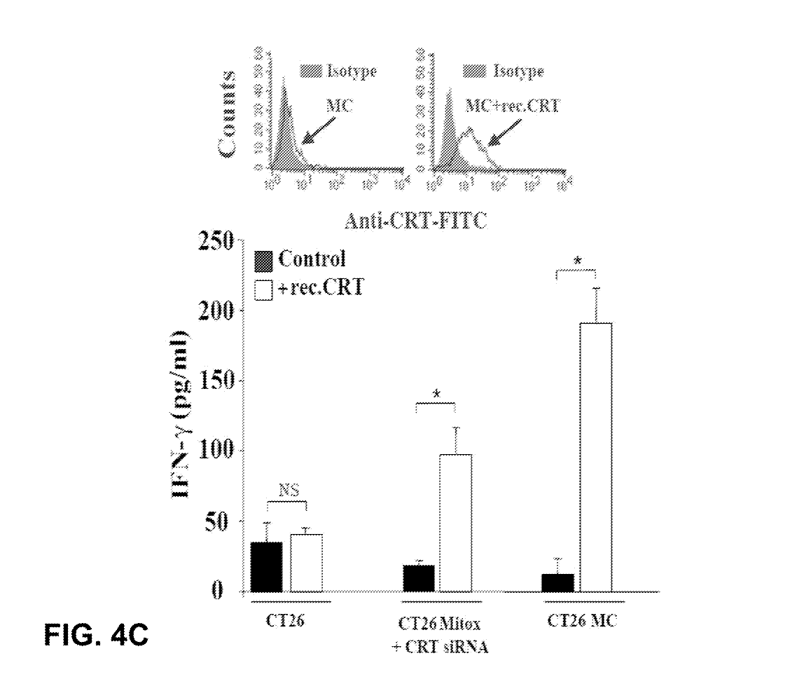

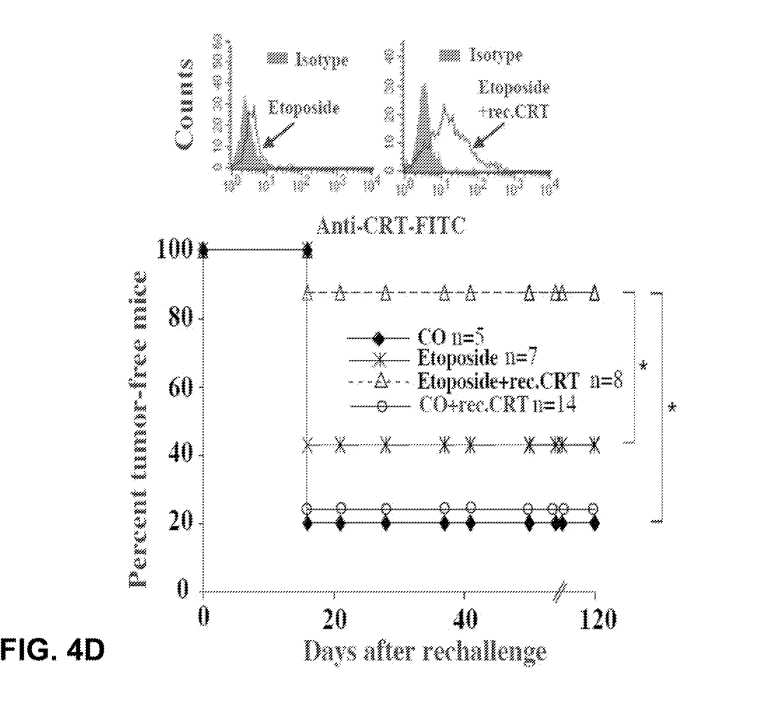

The knock-down of CRT compromised the immunogenicity of mitoxontrone-treated CT26 cells, and this defect was restored when rCRT was used to complement the CRT defect induced by the CRT-specific siRNA. This result was obtained in two distinct experimental systems, namely (i) when CT26 tumor cells were injected into the flank of Balb/c mice (or MCA205 cells were injected into C57Bl/6 mice) to assess the efficacy at anti-tumor vaccination (FIG. 4A) and (ii) when the tumor cells were injected into the foot pad to measure interferon-γ production by T cells from the popliteal lymph node (FIG. 4B). In this latter system, absorption of rCRT to the plasma membrane surface greatly enhanced the immunogenicity of cells that usually fail to induce an immune response such as mitomycin C-treated cells (FIG. 4C). Similarly, etoposide-treated cells coated with rCRT elicited a vigorous anti-tumor immune response in viva, in conditions in which sham-coated cells treated with etoposide were poorly immunogenic (FIG. 4D). However, absorption of rCRT to the cell surface without prior treatment with cell death inducers failed to elicit an anti-cancer immune response and live rCRT-pretreated cells inoculated into mice formed tumors, both in immunocompetent and immunodeficient mice.

In contrast, the knock-down of ERP57 compromised the immunogenicity of mitoxantrone-treated CT26 cells, and this defect was restored when rCRT was used to complement the CRT defect induced by the ERP57-specific siRNA. This defect was not restored when rERP57 was used to complement the CRT defect induced by the ERP57-specific siRNA. This result was obtained in two distinct experimental systems, namely (i) when CT26 tumor cells were injected into the flank of Balb/c mice (or MCA205 cells were injected into C57Bl/6 mice) to assess the efficacy of anti-tumor vaccination and (ii) when the tumor cells were injected into the foot pad to measure interferon-γ production by T cells from the popliteal lymph node. Thus, CRT surface translocation and not ERP57 critically determines the immunogenicity of cell death in vivo but do not determine cell death as such.

With regard to FIG. 4A, in vivo anti-cancer vaccination depends on CRT and not ERP57. CT26 transfected with siRNA specific for ERP57 and then treated with rCRT and/or mitoxantrone. The anti-tumor response was measured by simultaneously challenging BALB/c mice with mitoxontrone treated tumor cells in one flank and untreated, live tumor cells in the opposite flank. This addition of recombinant rCRT restores the protection against tumors.

With regard to FIG. 4C, CT26 cells lacking CRT expression after depletion of CRT with a siRNA and mitoxantrone treatment and exogenous rERP57 applied and then injected into the food pad, followed by assessment of the IFN-γ secretion by cells from the draining lymph nodes. The addition of recombinant ERP57 did not restore the protection against tumors either the secretion of IFN-γ.

### Example 5

### Inhibitors of PP1/GADD34 Induce both CRT and ERP57 Exposure and Induce Immunogenicity

Since anthracyclines-induced CRT and ERP57 exposure was a rather rapid process (within 1 hour, FIG. 1SA, 1SB, 2SC, 2SD), it was suspected that anthracyclines might exert effects that are not mediated by genotoxic stress. In response to mitoxantrone, enucleated cells (cytoplasts) readily (within 1 hour) exposed both CRT (FIG. 5A) and ERP57, and became preys of DC as efficiently as intact cells (FIG. 3A), indicating the existence of a cytoplasmic (non-nuclear) anthracyclines target. Anthrocyclines failed to induce immediate mitochondrial stress, yet caused the rapid phosphorylation of elF2α (FIG. 5B), a protein that is typically hyperphosphorylated in ER stress due to the activation of stress kinases. Knock-down of the four kinases known to phosphorylate elF2α (GCN2, HRI, PERK, PKR) failed to inhibit the anthracyclines-stimulated CRT and ERP57 exposure. In contrast, knock-down of either GADD34 or the catalytic subunit of protein phosphatase 1 (PP1) (FIG. 5C), which together form the PP1/GADD34 complex involved in the dephosphorylation of elF2α was sufficient to induce both CRT (FIG. 5D) and ERP57 exposure. Both CRT and ERP57 exposure triggered by PP1 or GADD34 depletion was not further enhanced by mitoxantrone (FIG. 5D), suggesting that PP1/GADD34 and anthracyclines act on the same pathway to elicit CRT and ERP57 translocation to the cell surface. CRT and ERP57 exposure was efficiently induced by chemical PP1/GADD34 inhibitors, namely tautomycin, calyculin A (which both inhibit the catalytic subunit of PP1), as well as by salubrinal (which inhibits the PP1/GADD34 complex) (FIG. 5E). All these PP1/GADD34 inhibitors induced CRT exposure with a similar rapid kinetics as did anthracyclines, both in cells (FIG. 5E, 2SG) and in cytoplasts.

Mitoxantrone and salubrinal induced CRT and ERP57 exposure on a panel of tumor cell lines from murine (MCA205, B16F10. J558) or human origin (HeLa, A549, HCT116). Both CRT and ERP57 exposure induced by anthracyclines and PP1/GADD34 inhibitors was not affected by inhibitors of transcription, translation or microtubuli, yet was abolished by latrunculin A, an inhibitor of the actin cytoskeleton and exocytosis (FIG. 3SE, 3SF).

Inhibition of the PP1/GADD34 complex with salubrinal, calyculin A or tautomycin was not sufficient to induce immunogenic cell death (FIG. 5F, G) (and the cells, which did not die, formed lethal tumors when injected into animals). However, these inhibitors greatly enhanced both CRT and ERP57 exposure (FIG. 5F) and the immunogenic potential of cells succumbing to etoposide (FIG. 5G) or mitomycin C. This immunostimulatory effect was abrogated by knocking down CRT (FIG. 5G). Altogether, these results demonstrate that PP1/GADD34 inhibition induces both CRT and ERP57 exposure, which, in turn, can stimulate the anti-tumor immune response.

### Example 6

### Inhibitors of PP1/GADD34 by Specific Peptide Induce both CRT and ERP57 Exposure

CT26 treated with the peptide inhibitor increase greatly and quickly (1 h after the treatment) the CRT and ERP57 exposure (FIGS. 5SB, 5SC). This exposure was stable and independent from the time of treatment. The peptide inhibitor induced CRT and ERP57 exposure with a similar level and rapid kinetics as did anthracyclines and the chemical inhibitors of PP1/GADD34 inhibitors. The peptide had no toxic effect and did not increase the percentage of dead cells positives for staining with the vital dye DAPI and the PS-binding dye Annexin V after 24 h of treatment (FIG. 5SA).

### Example 7

### Immunogenic Chemotherapy by In Vivo Application of CRT or PP1/GADD34 Inhibitors

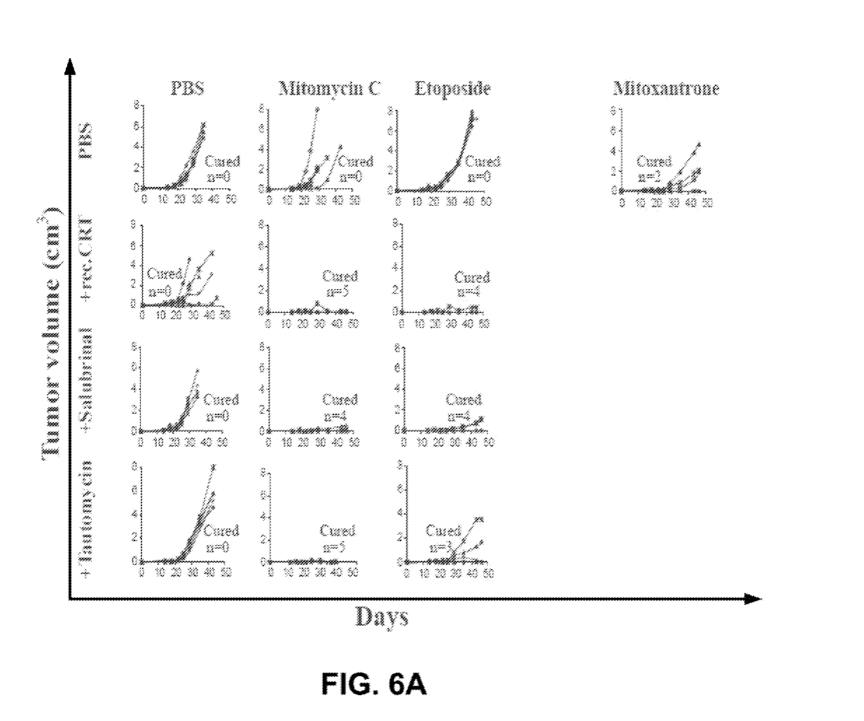

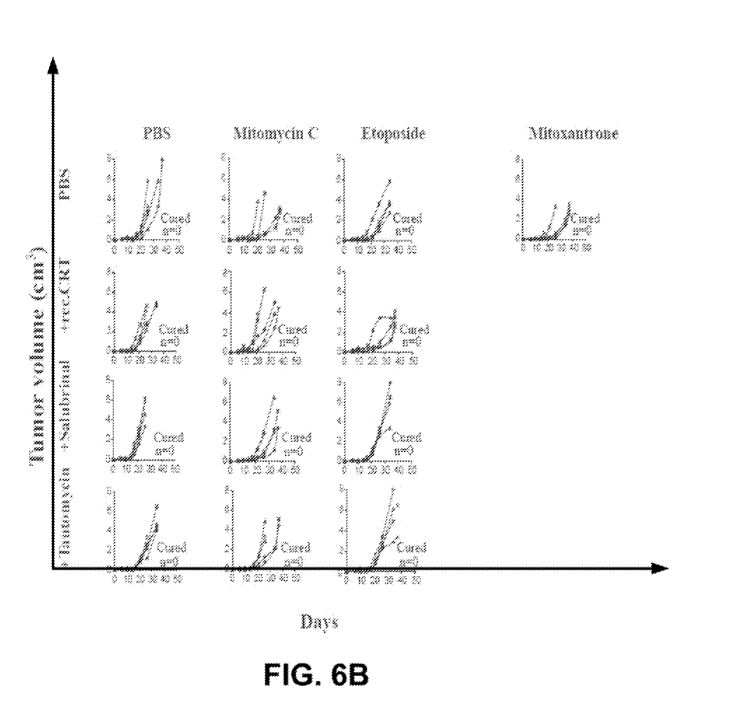

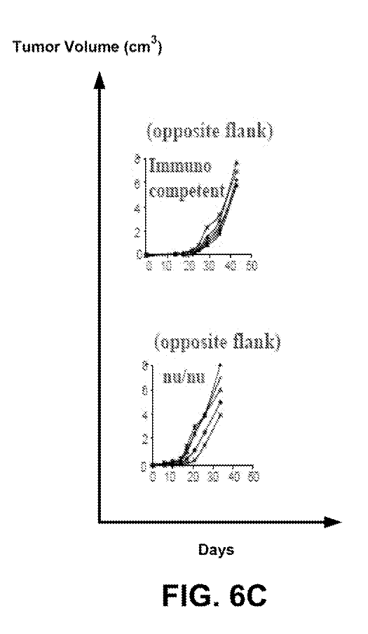

A single intratumoral injection of mitoxantrone into established 14-day-old CT26 tumors was able to cause their permanent regression in some but not all cases, if the tumors were established in immunocompetent BALB/c mice (FIG. 6A). However, there was no cure by mitoxontrone if the tumors were carried by immunodeficient nu/nu mice (FIG. 6B). The intratumoral injection of rCRT, salubrinal, tautomycin, etoposide or mitomycin C had no major therapeutic effect, neither in immunocompetent nor in nu/nu mice. However, the combination of a cell death inducer (etoposide or mitomycin C) plus rCRT was able to cause tumor regression, in immunocompetent (but not in immunodeficient) animals. To obtain a therapeutic effect, rCRT had to be injected into the tumor.

rCRT injected into a distant site did not ameliorate the antitumoral effects of intratumorally injected etoposide (FIG. 6C). Similarly, etoposide or mitomycin C could be combined with drugs that induce CRT exposure (salubrinal or tautomycin), leading to stable disease or complete tumor regression in immunocompetent (but not in athymic) hosts (FIG. 6A, B). Live CT26 cells failed to grow in animals that had been cured from CT26 tumors, indicating the establishment of a permanent anti-tumor immune response. Similar results were obtained when established MCA205 sarcomas (in C57Bl/6 mice) or PRO colon carcinomas (in BDIX rats) were treated by local injections of weakly immunogenic cell death inducers plus rCRT or PP1/GADD34 inhibitors. These results delineate a strategy of immunogenic chemotherapy for the cure of established cancer.

Fertilization

In mammals, sperm-eggs interaction is based on molecular events either unique to gametes or also present in somatic cells. In gamete fusion, it is unknown which mechanism is gamete specific and which mechanism is shared with other systems. Membrane fusion is an important phenomenon that occurs in different biological systems such as the entry of enveloped virus into cells, cellular trafficking, endocytosis and exocytosis, osteoclasts, and myotube formation, and fertilization. Cellular membrane do not fuse spontaneously, and specific fusion proteins tightly control membrane fusion events through interaction with lipids and others proteins. Because fusions proteins active in a cell-cell fusion have not yet been identified, there currently exists no specific information about the involvement of colreticulin and ERP57 in the sperm-eggs fusion. The present invention teaches that the gamete fusion is dictated by the membrane exposure of CRT and also requires a sperm surface-associated disulfide isomerase activity.

Results

### Example 1

### Calreticulin and ERP57 Exposure Occurs in Capacitated Sperms

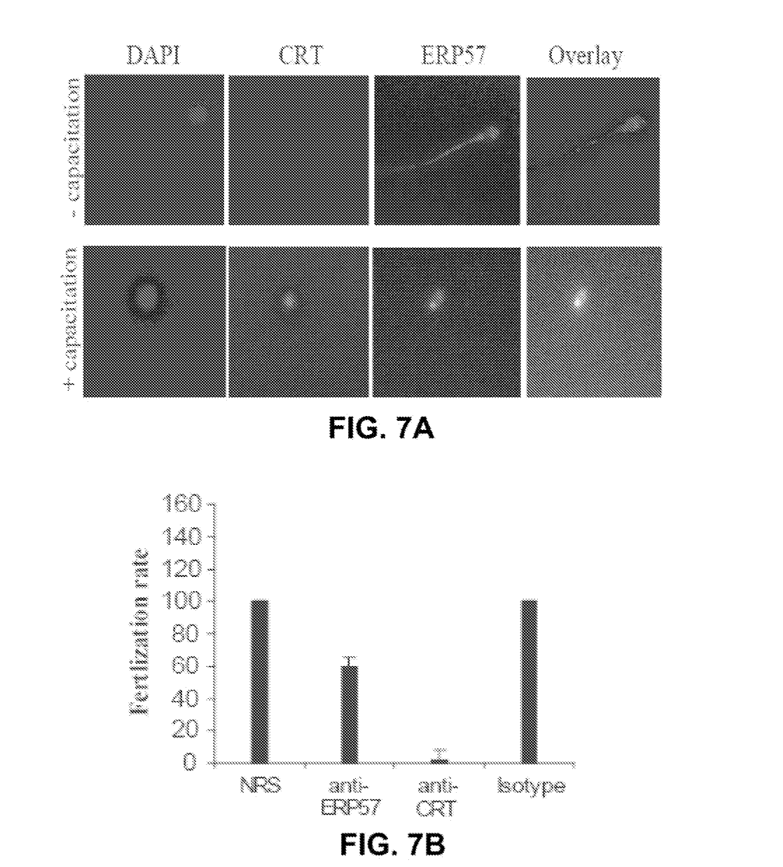

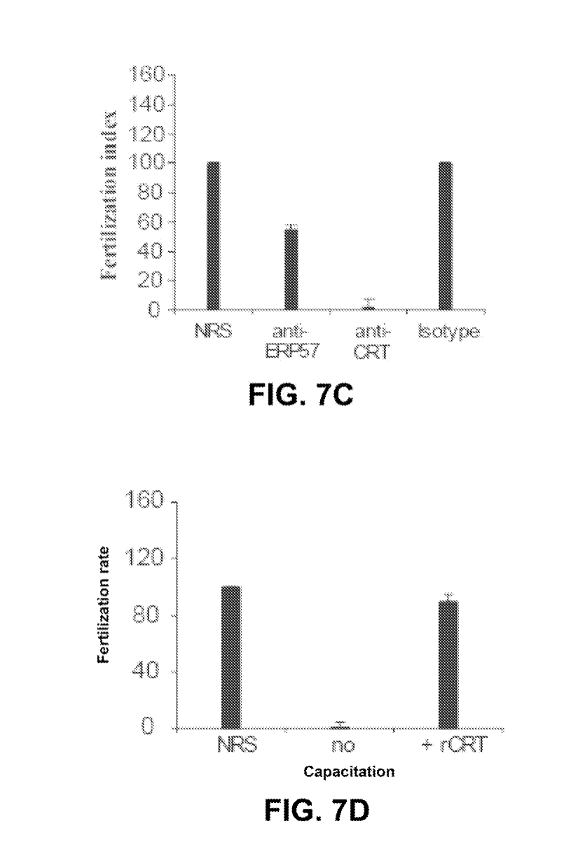

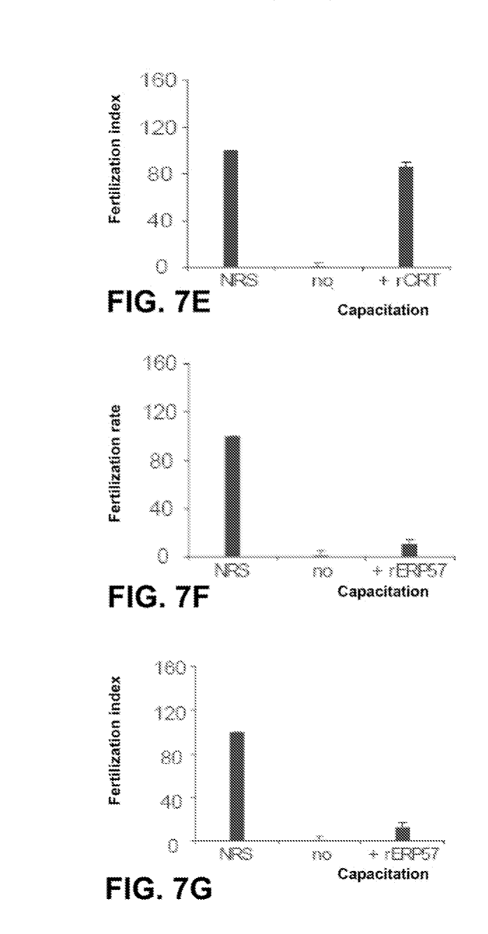

In one experiment according to the present invention, CRT surface exposure was detectable by immunofluorescence staining on capacitated sperm (FIG. 7A): otherwise, this exposure was absent on non-capacitated sperm. The surface expression of CRT was colocalized with surface ERP57 on capacitated sperm. The surface expression of ERP57 on capacitated sperm was concentrated in the head of the sperm in contrast to non-capacitated sperm where the ERP57 is more dispatched (FIG. 7A).

### Example 2

### Calreticulin Exposure Dictates Sperm-Egg Fusion

The incubation of capacitated sperm with blocking antibody to calreticulin, produces a total inhibition of fusion; otherwise, the blocking of ERP57 produces a partial, but significant, inhibition of fusion (FIG. 7B-7C). No effect was obtained with the control isotype (FIGS. 7B-7C). In contrast to sperms, the blocking of CRT or ERP57 on the surface of eggs had no effect on the fusion. Moreover, the absorption of recombinant CRT to the cell surface of sperm without prior capacitation restores very efficiently the fusion sperm-eggs (FIG. 7D-7E), in contrast to rCRT, the recombinant ERP57 had no significant effect on the fertilization rote and index (FIGS. 7F-7G).

Materials and Methods

Gamete Isolation. Using mature, cumulus-free oocytes from superovulated 6-week-old to 8-week-old ICR female mice, eggs were denuded from the zona pellucida and loaded with DAPI. Sperms were collected from each cauda epididymis and vas deferens of 10- to 12-week-old ICR males. Sperms were allowed to disperse in a 500 μl of M199 containing 3% BSA, and then diluted 1:10 in 500 μl of M199, 3% BSA and incubated for 3 hr at 37° C. and 5% CO₂. During this incubation, the sperms acquired the capacity to fertilize an egg and are thus termed “capacitated”. All the experiments were performed according to the animal care and use protocols approved by French and European Union Ethical Committee.

Sperm-Egg Fusion Assay. Capacitated sperms or eggs were incubated with blocking antibody to CRT (ABR bioaffinity bioreagents), blocking antibody to ERP57 (Abcam), or isotype control for 30 min at 37° C. and 5% CO₂. After the incubation, the sperms or eggs were washed. Sperms were added at a final concentration of 1-3×10⁵ sperm/ml and coincubated with gamete for 40 min at 37° C. and 5% CO₂. The oocytes were then washed to release loosely bound sperms and mounted onto microscope slides. Alternatively, non-capacitated sperms were incubated with recombinant CRT or ERP57, 3 μg/10⁵ sperm, for 30 min 37° C. and 5% CO₂, followed by three washes with PBS. Sperm-egg fusion scored by the fluorescent labeling of sperm nuclei by DAPI transferred from preloaded eggs. Fertilization rate (FR) is the percentage of oocytes with at least one fused sperm, and Fertilization index (FI) is the mean number of fused sperm per egg. Both the FR and FI are expressed of the control treatment.

Sperm Immunofluorescence. The sperms were capacitated for 3 hours and then incubated with antibody specific to CRT (Abcam), ERP57 (Abcom) or normal rabbit serum at a 1:200 dilution for 30 min at 37° C. and 5% CO₂. The sperms were then layered on top of 1 ml of M199, 3% BSA, and centrifuged for 3 minutes at 3000 rpm, resuspended in PBS, and fixed in 2% PFA (paraformaldehyde) for 5 minutes at 4° C. After three washes with PBS, the sperms were incubated with the anti-rabbit IgG (H+L) Alexa fluor 488-conjugates or 594-conjugates (1:500) in PBS at 4° C. for 30 min. After washing three times with PBS, the sperms were layered on top of 1 ml of M199, 3% BSA, centrifuged, and then resuspended in PBS and mounted on glass slides.

Statistical analyses Statistical differences between the groups were analyzed using student's t-test.

Transplantation Rejection in Mammals

The teachings of the present invention may also be used to the determination of transplantation rejection in mammals, by detecting the level of plasma membrane CRT and ERP57. Moreover, the use of blocking CRT and ERP57 antibodies, and inhibitory competitive peptide of plasma membrane CRT and ERP57, as described herein, allows the acceptance and toleration of the transplantation.

Exemplary Applications

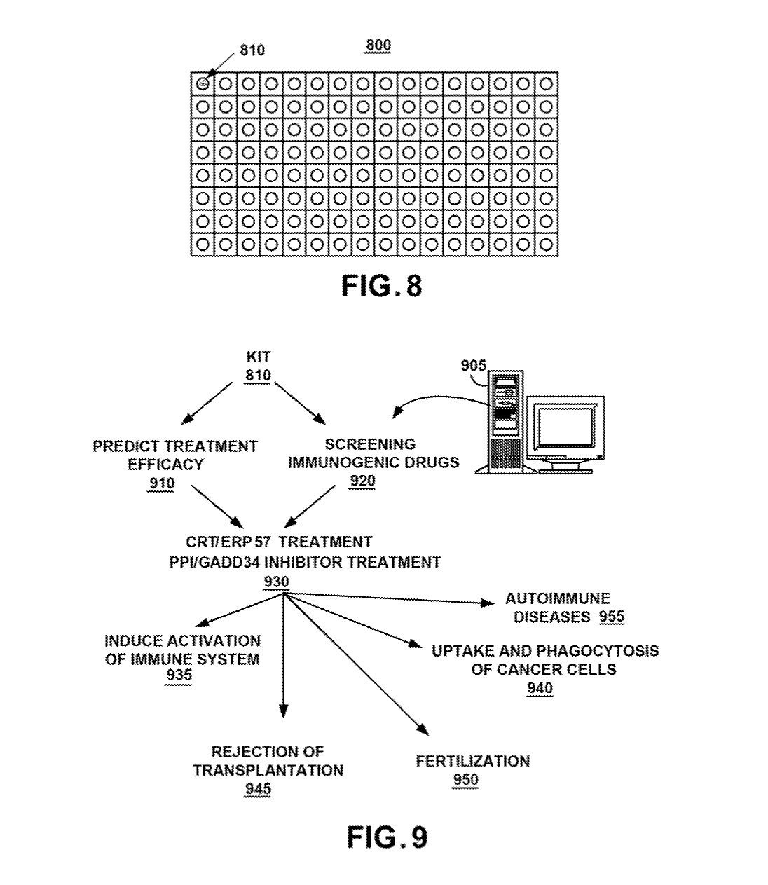

FIG. 8 illustrates a test kit or test chip 800 (also referred to herein as the kit 800) for use in the implementation of the present invention. The kit 800 contains several compartments (or vials) 805, that comprises one or more containers or compartments 805 filled with one or more of the compounds or ingredients of the pharmaceutical compositions 810 of the present invention. In one embodiment, the kit 800, referred to as the CRT kit, contains calreticulin antibodies. In another embodiment, the kit 800, referred to as the ERP57 kit, contains ERP57 antibodies.

The kit 800 is provided, as described herein, for the diagnosis and/or the treatment of pathological conditions (such as cancers), or for the practice of any of the screening or diagnosis methods described herein.

In one embodiment, the test kit or test chip 800 contains at least one of the compounds (or compositions) 810 described herein for the detection of the proteins calreticulin and ERP57, at the cell surface, according to the methods described herein.

The compounds 810 may, if desired, be presented in a pack or dispenser device which contains one or more unit dosage forms containing the active ingredient or protein described herein. The pack may for example comprise metal or plastic foil, such as a blister pack. The pack or dispenser device may be accompanied by instructions for administration.

When the kit is used for screening or testing new drugs (e.g., immunogenic molecules and/or compounds as described herein), for fertility screening, or for other fertility screening tests, the tests are carried out in a laboratory and the drug contained in the kit, is not injected in the patient.

The invention also provides a kit 800 for carrying out the therapeutic regimens of the invention. Such kit 800 comprises in one or more containers, therapeutically effective amounts of the protein described herein, in a pharmaceutically acceptable form and/or the peptide inhibitor of the complex PP1/GADD34 and/or any other inhibitor of the complex PP1/GADD34 as described herein. The magnitude of a therapeutic dose of the compound will vary with the severity of the condition to be treated and the route of administration.

In another embodiment, the kit 800 further comprises a needle or syringe, preferably packaged in sterile form, or any other method and way of injection, for injecting the compound 810. The frequency of administration of the compound of the present invention varies with the patient or recipient. As an example one administration may be sufficient for certain mammals, while additional administrations may be required for other mammals.

FIG. 9 illustrates an overall method 900 for the implementation of the various methods of the present invention. Method 900, or parts thereof, may be implemented by a processor 905 by means of a computer program product that includes a plurality of sets of instruction codes for automatically carrying out the various steps of the methods described herein.

Method 900 is initiated with the use of the kit 800 that enables the prediction of the efficiency of the treatment according to the teachings herein, prior to the commencement of the treatment (step 910). Alternatively, the kit 800 will assist in the screening of immunogenic drugs or medications prior to extensive testing (step 920). As an example, if a candidate drug induces the translocation of calreticulin or/and ERP57, it would be deemed to be efficient; otherwise, it is not efficient.

The present invention a method of detecting the calreticulin and/or ERP57 at the cellular surface for the screening of direct or indirect immunogenic drugs. Such screening method comprises detecting the calreticulin and/or ERP57 protein at the cell surface, and uses anti calreticulin antibodies and/or ERP57 antibodies for the screening of direct or indirect immunogenic drugs. The screening of direct and indirect immunogenic drugs could lead to the identification of more efficient anti-tumorous agents and new efficient molecules, for use in the treatment of mammal diseases and health-related conditions.

If at step 910 it determined that the particular treatment would be sufficiently effective for the treatment of the condition in question, then treatment (such as cancer treatment) is commenced at step 930, by inducing the activation of the immune system as described herein (step 935). Alternatively, the treatment includes the uptake and destruction of the affected cells, such as cancerous cells (step 940).

According to another embodiment of the present invention, the treatment could be the determination of the probability of rejection of an organ transplant or graft (step 945), the probability of success of the fertilization process (step 950), and/or to treat and detect autoimmune diseases (step 955).

It is to be understood that the specific embodiments of the invention that have been described are merely illustrative of certain application of the principle of the present invention. Numerous modifications may be made to the description herein, without departing from the spirit and scope of the present invention. For example, the low expression of CRT and/or ERP57 protein or the deletion of the CRT and/or ERP57 gene provide a bad prognostic factor, and are thus indicative of a probability or a propensity of a patient disposition to a health condition in question, such as cancer.

What is claimed is:

1. A pharmaceutical compound for use in the treatment of a health condition in a mammal, comprising:
an antibody that interferes with an increased translocation of a protein, leading to an increased immunogenicity of cell death.

2. The pharmaceutical compound of claim 1, wherein the antibody comprises anti-calreticulin.

3. The pharmaceutical compound of claim 1, wherein the antibody comprises anti-ERP57.

4. The pharmaceutical compound of claim 1, wherein the antibody comprises a blocking antibody.

5. The pharmaceutical compound of claim 1, wherein the antibody comprises a neutralizing antibody.

6. The pharmaceutical compound of claim 2, wherein the protein comprises a calreticulin protein.

7. The pharmaceutical compound of claim 1, wherein the protein comprises an ERP57 protein.

8. The pharmaceutical compound of claim 1, wherein the health condition includes an autoimmune disease.

9. The pharmaceutical compound of claim 8, wherein the autoimmune disease includes SLE.

10. The pharmaceutical compound of claim 8, wherein the autoimmune disease includes rheumatoid arthritis.

11. The pharmaceutical compound of claim 8, wherein the autoimmune disease includes dermatitis.

12. The pharmaceutical compound of claim 1, wherein the health condition includes an autoimmune disease.

13. The pharmaceutical compound of claim 1, wherein the health condition includes an allergy.

14. The pharmaceutical compound of claim 1, wherein the health condition includes a graft versus host disease.

15. The pharmaceutical compound of claim 1, wherein the health condition includes transplant rejection.

16. A pharmaceutical compound for use in the treatment of a health condition in a mammal, comprising:
an peptide that interferes with an increased translocation of a protein, leading to any of: an increased immunogenicity of cell death, autoimmune disease, or transplantation rejection.

17. The pharmaceutical compound of claim 16, wherein the peptide comprises an inhibitory peptide.

18. The pharmaceutical compound of claim 16, wherein the peptide comprises a competitive peptide.

19. The pharmaceutical compound of claim 16, wherein the protein comprises a calreticulin protein.

20. The pharmaceutical compound of claim 17, wherein the protein comprises an ERP57 protein.
